(19) United States
(12) Patent Application Publication
(10) Pub. No.: US 2001/0039666 A1
(43) Pub. Date: Nov. 8, 2001

(54) NON-HUMAN MAMMALIAN MODEL FOR ATHEROSCLEROSIS AND METHODS FOR SCREENING AGENTS FOR USE IN THE TREATMENT OF ATHEROSCLEROSIS

(76) Inventors: DAVID A. DICHEK, HILLSBOROUGH, CA (US); DARREN SCHNEIDER, SAN FRANCISCO, CA (US); GIUSEPPE VASSALLI, CULLY (CH)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/229,399

(22) Filed: Jan. 11, 1999

(51) Int. Cl. A01K 67/00; A61K 48/00; C12N 15/63; C12Q 1/70

(52) U.S. Cl. 800/3; 424/93.1; 435/325; 435/1.1; 435/455; 435/7.1; 435/5

(57) ABSTRACT

The present invention relates generally to methods of screening test agents for an activity on atherosclerosis. In particular, the present invention relates to methods of screening a test agent for an activity on atherosclerotic lesion development in an animal and an animal model of atherosclerosis. The present invention also relates generally to methods of screening a gene for a therapeutic or prophylactic activity on atherosclerotic lesion development in an animal.

### FIELD OF THE INVENTION

The present invention relates generally to methods of screening agents for an activity on atherosclerosis and an animal model of atherosclerotic disease.

### BACKGROUND OF THE INVENTION

Atherosclerosis is a major cause of morbidity and mortality throughout the world. Atherosclerosis causes heart attacks, strokes, angina and other forms of heart disease, loss of limb function, renal failure, and sudden death. Atherosclerosis is a progressive disease which affects native human blood vessels (e.g., arteries) beginning in childhood and adolescence and typically produces clinical manifestations by middle to late adulthood. See generally, E. Braunwald, HEART DISEASE: A TEXTBOOK OF CARDIOVASCULAR MEDICINE (3d ed. 1988) and E. Braunwald, HEART DISEASE: A TEXTBOOK OF CARDIOVASCULAR MEDICINE (5th ed. 1996), each of which is incorporated herein by reference in its entirety for all purposes. Morbid sequelae of atherosclerosis generally become manifest during the 6th to 7th decades of an individual's life. Atherosclerosis also affects vascular grafts (such as those used in heart bypass operations) with a more aggressive course, resulting in impairment of function of the majority of bypass grafts within 10 to 15 years. Reoperation to replace these diseased grafts is often a hazardous and morbid undertaking. Given the dangers and widespread occurrence of atherosclerosis, prevention and education regarding the disease has been one of the primary goals of health officials throughout the world.

The pathogenesis of atherosclerosis, particularly at the local level (such as within arterial wall), is not well understood. To determine the pathogenesis of atherosclerosis and related vascular diseases and to develop effective strategies for the prevention and treatment of such diseases in humans, it is imperative to have a satisfactory animal model system of atherosclerosis. Early efforts at producing animals having atherosclerotic disease representative of human atherosclerotic disease have been problematic. One animal model presently in use for the study of atherosclerosis, for example, employs balloon arterial injury procedures with feeding of cholesterol to the animal to induce a lesion. Unfortunately, the lesions produced in this manner are not representative of human atherosclerotic lesions. The balloon arterial injury technique significantly injures the artery, causing denuding of the endothelium of the artery and causing detrimental effects to the arterial wall. Weidinger et al., Circulation 84:755-767 (1991); Faxon et al., Arteriosclerosis 2:125-133 (1982). The endothelium comprises a layer of flat cells lining blood and lymphatic vessels—as well as the heart. Denudation removes the protective layer of endothelium from the underlying surface. Such injuries are not observed in human atherosclerotic lesions. Human atherosclerotic lesions have a histologically intact endothelium. Moreover, with such an animal model, screening or investigation of the effect of a genetic or pharmacologic agent on atherosclerotic lesion development is not possible, because the agent's effect can be tested only after the balloon-artery induced lesion has been established.

Another animal model alleged to represent human atherosclerosis is obtained by feeding the animal a diet high in cholesterol. Lesions produced in the vessels of such animals are characterized by the presence of large quantities of macrophages, with few or no smooth muscle cells (SMC). In contrast, in human atherosclerotic lesions, the intimas of the vessels usually contain numerous smooth muscle cells and T cells—in addition to macrophages. The intima is the innermost layer of the wall of the blood vessel (e.g., lymphatic vessel, artery, or vein).

In presently known animal models of atherosclerosis, it is not possible to investigate the effects of agents, such as genes, that act locally at the level of the vessel wall. Existing animal models of atherosclerosis cannot be used to elucidate the role of gene expressed primarily in the artery wall or at the lesion site. Present mouse models, for example, are inadequate because they either overexpress a gene (resulting in widespread systemic expression in the blood and elsewhere—as well as in vessels) or constitute genetic “knockout” models which do not express the desired gene. It would be of benefit to have an animal model of atherosclerosis in which the effects of genes that are expressed locally at a specific site in the arterial wall can be investigated.

In the absence of an optimal animal model for atherosclerosis in which vessel wall gene expression can be directly manipulated, the identification of local, specific pathogenic processes involved in atherosclerotic lesion development and approaches for treating and preventing atherosclerosis have been greatly hindered. Further, without appropriate animal models, methods for screening agents for their potential therapeutic or prophylactic effects on atherosclerosis have been severely thwarted. Notably, although some procedures have proved effective in reducing the effects of existing atherosclerotic conditions in a subject (e.g., balloon and laser angioplasty have been found to be effective in minimizing plaque deposits and restoring blood flow through diseased blood vessels, such as arteries), no known procedures (delivered directly to vascular tissue) have been found to be effective in preventing atherosclerosis.

Accordingly, there is a need for an animal model of atherosclerosis which more closely represents human atherosclerosis than do currently existing animal models and methods for screening agents for their potential therapeutic and prophylactic effects on atherosclerosis using such models. It would also be worthwhile to have available prophylactic and therapeutic treatment methods to prevent and treat the disease. The present invention fulfills this and other needs.

### SUMMARY OF THE INVENTION

The present invention relates generally to methods of screening a test agent for an activity on atherosclerotic lesion development in a non-human mammal. Such methods comprise: (a) administering to the non-human mammal an atherogenic diet; (b) isolating a blood vessel of the non-human mammal; (c) delivering or introducing a proinflammatory agent to the blood vessel of the non-human mammal; (d) delivering the test agent to the non-human mammal; and (e) monitoring a property of the blood vessel to indicate an activity on atherosclerotic lesion development in the blood vessel.

In another aspect, the invention provides methods of screening a gene for a therapeutic or prophylactic activity on atherosclerotic lesion development in a non-human mammal, which comprises: (a) administering an atherogenic diet to the non-human mammal; (b) isolating a blood vessel of the non-human mammal; (c) delivering or introducing a vector to the blood vessel, wherein the vector comprises a gene and expresses the gene in the blood vessel; and (d) monitoring a property of the blood vessel which indicates a therapeutic or prophylactic activity of the gene on atherosclerotic lesion development in the blood vessel.

The present invention also provides methods of producing a non-human mammalian model of atherosclerosis. Such methods comprise: (a) administering an atherogenic diet to the non-human mammal; (b) isolating a blood vessel of the non-human mammal; (c) delivering or introducing a proinflammatory agent to the blood vessel of the non-human mammal; and (d) maintaining the non-human mammal for a time sufficient for an atherosclerotic lesion to develop in the blood vessel, thereby producing a model of atherosclerosis.

In yet another aspect, the invention provides non-human mammalian models for atherosclerotic disease. Such models comprise a non-human mammal having a blood vessel that is characterized by having an atherosclerotic lesion, wherein the blood vessel has a histologically intact endothelium and an intima comprising smooth muscle cells and macrophages, and the lesion comprises or contains a proinflammatory agent.

Also provided are methods of inducing development of an atherosclerotic lesion in a non-human mammal. Such methods, which are useful for producing experimental atherosclerotic lesions for study and analysis, comprise: (a) administering to the non-human mammal an atherogenic diet; (b) isolating a blood vessel of the non-human mammal; and (c) introducing or delivering a proinflammatory agent into the blood vessel, wherein said administering to the non-human mammal of the atherogenic diet and said introducing or delivering of the proinflammatory agent into the blood vessel of the non-human mammal induces, causes, or promotes development of an atherosclerotic lesion in the blood vessel.

In another aspect, the present invention provides methods of gene therapy for prophylactic or therapeutic treatment of atherosclerosis in a subject in need of such treatment. Such methods comprise delivering or introducing a vector to a blood vessel of the subject, wherein said vector comprises a gene which encodes a therapeutically or prophylactically useful peptide or protein for atherosclerosis, said gene being expressed in the subject, thereby promoting prophylactic or therapeutic treatment of atherosclerosis in the subject.

A further understanding of the nature and advantages of the inventions herein may be realized by reference to the detailed description of the specification and the associated drawings.

### BRIEF DESCRIPTION OF THE FIGURES

FIG. 1 is a schematic illustration of methods of the invention regarding producing animal models of atherosclerosis and screening agents for their anti-atherogenic or proatherogenic activities on atherosclerotic lesion development. In the illustrated experiments, subject rabbits were fed either a standard (normal) diet consisting of High Fiber Laboratory Rabbit Diet #5326 (Dean's Feed, Belmont, Calif.) or an atherogenic diet (e.g., 0.25% cholesterol and 3% soybean oil) (i.e., high-cholesterol, high-fat diet) for 4 weeks. Following this period, a carotid artery in each such rabbit was surgically isolated and infused with: (a) a replication-deficient adenoviral vector expressing a biologically active gene (e.g., a replication-deficient adenoviral vector that expresses Fas ligand (termed “AdFasL” vector)), (b) a replication-deficient control adenoviral vector that did not comprise a gene (termed an “AdNull” vector)), or (c) a “vehicle” solution comprising a virus storage buffer (virus storage buffer comprises 10 mM Tris-HCl pH 7.4, 1 mM MgCl₂, and 10% glycerol) diluted in Dulbecco's Modified Eagle Medium (DMEM) (Biofluids, Inc., Rockville, Md.) containing 1 mg/ml rabbit serum albumin (Sigma Chemical Co., St. Louis, Mo.). The vehicle solution does not contain a vector or other proinflammatory agent. The vehicle solution comprises the virus storage buffer in DMEM with rabbit serum albumin. Rabbit carotid arteries were subsequently harvested at time points ranging from 2 to 28 days and examined using computer-assisted morphometry and immunohistochemical staining techniques, as described in more detail below. Infusion of the AdNull vector into rabbit carotid arteries resulted in the production of arterial atherosclerotic lesions of moderate size. The results with AdNull vector served as a control against which lesions generated with the experimental AdFasL vector were compared.

FIGS. 2A-2D represent four microscopic views of sections from rabbit carotid arteries. All vessels were stained with Movat's pentachrome stain, as described in detail below. These four views illustrate different morphologies of atherosclerotic lesions produced by infusion of either a vehicle solution or viral vector solution into a carotid artery of a hypercholesterolemic rabbit or a non-hypercholesterolemic rabbit. Hypercholesterolemia was induced in a rabbit by administering to the rabbit an amount of an atherogenic diet (or high-cholesterol or high-fat diet) sufficient to produce plasma cholesterol levels of at least 400 milligram/deciliter (mg/dl). A standard or normal diet (e.g., without excessive amounts of cholesterol or fat) was administered to non-hypercholesterolemic rabbits. Black arrows indicate the internal elastic lamina of the arteries. Magnification was ×100 for FIGS. 2A-2D. FIGS. 2A-2D were stained with Movat's pentachrome stain.

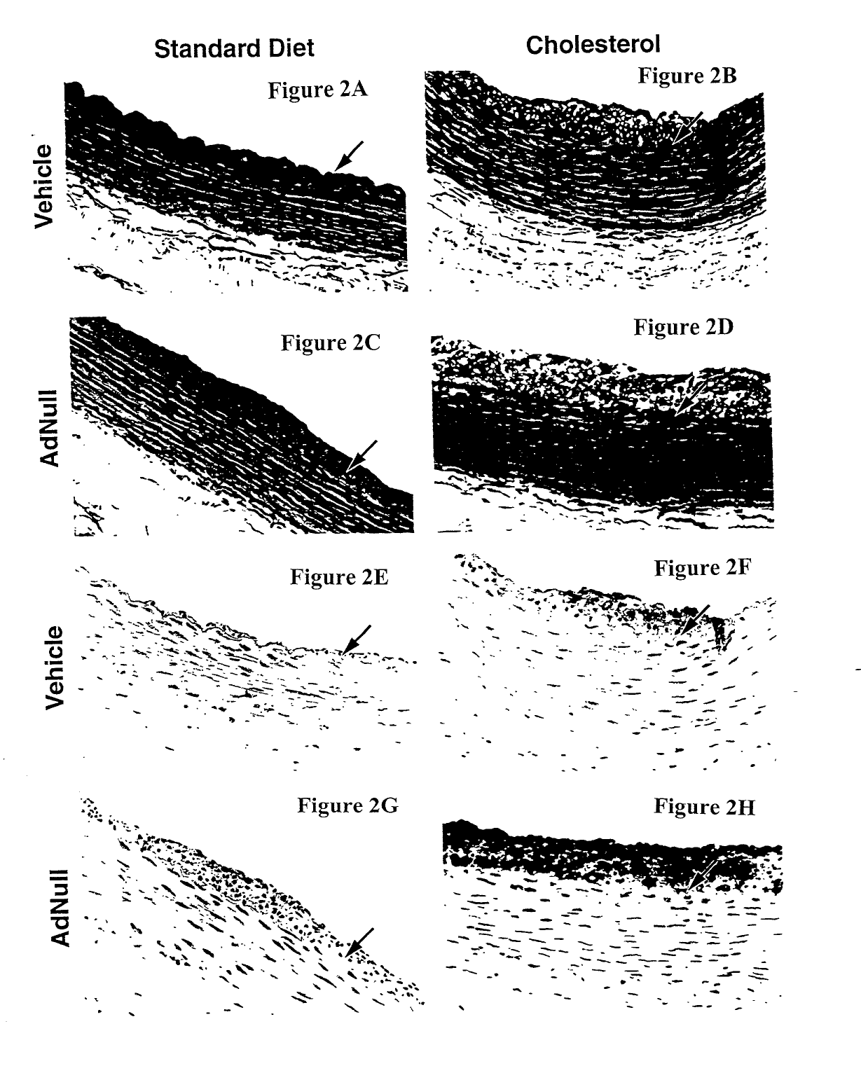

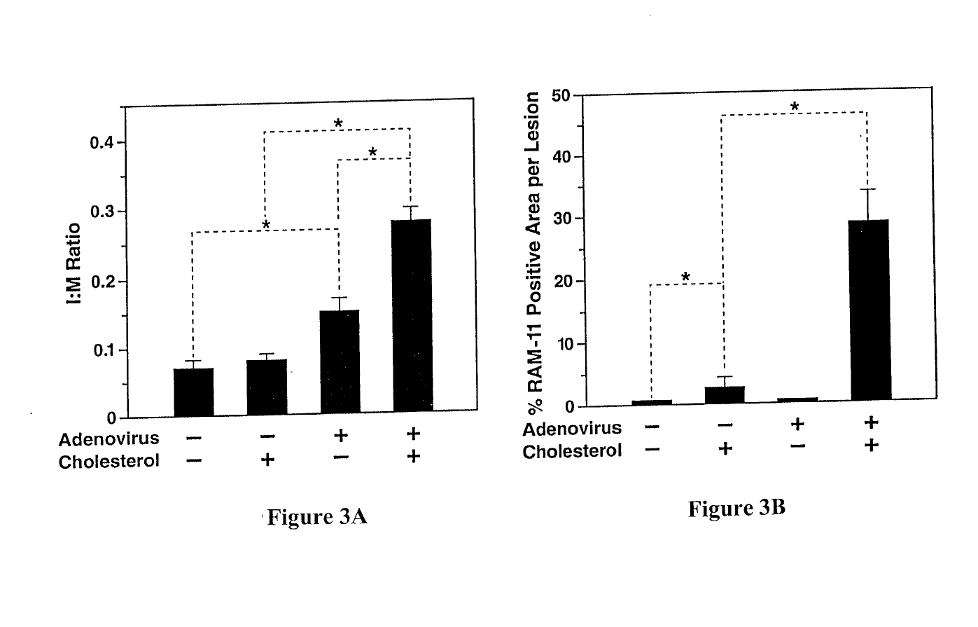

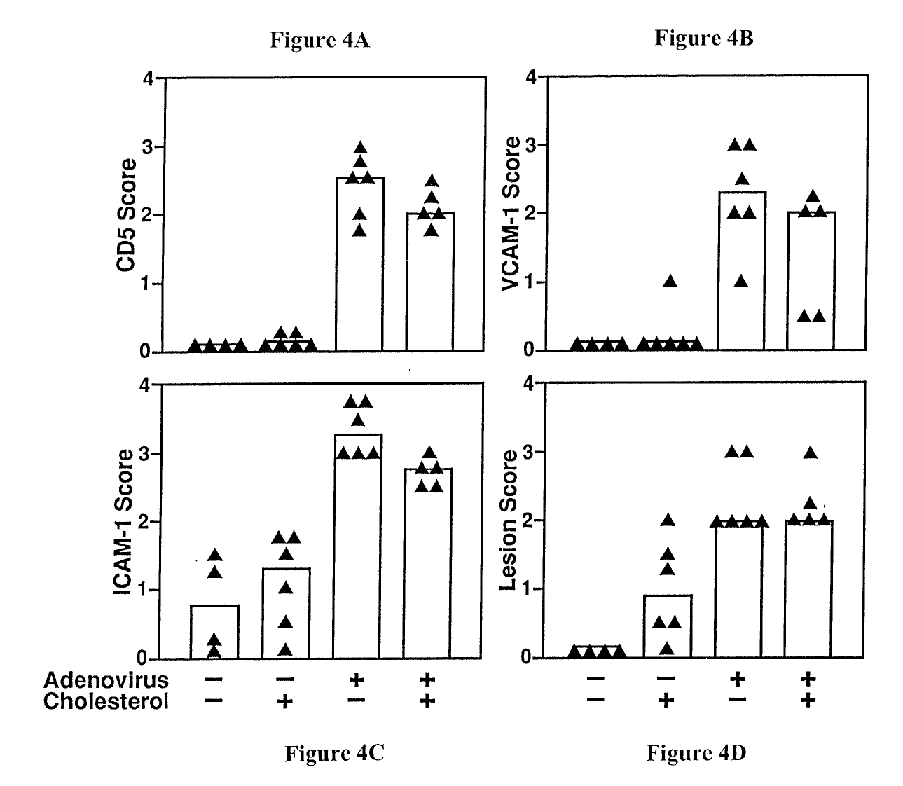

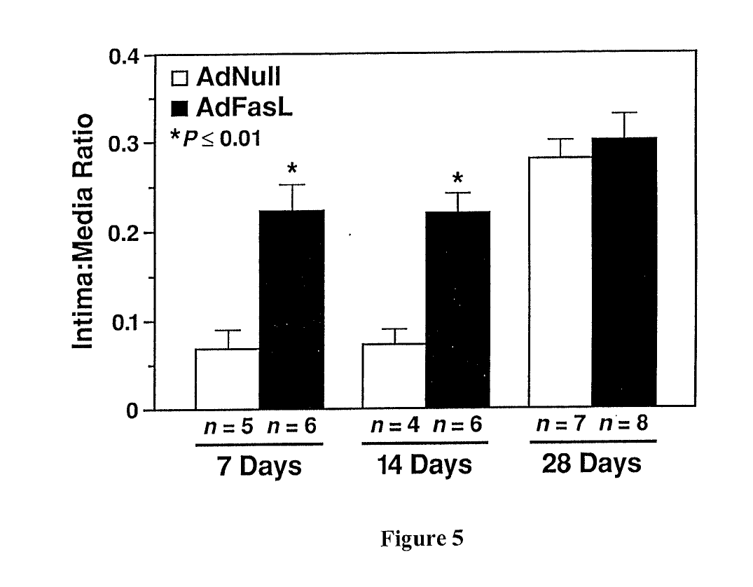

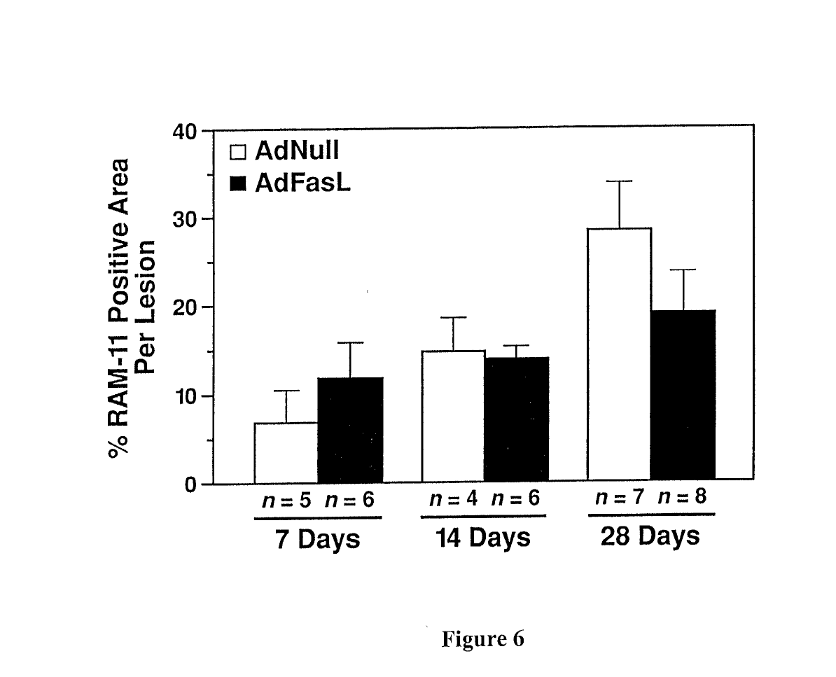

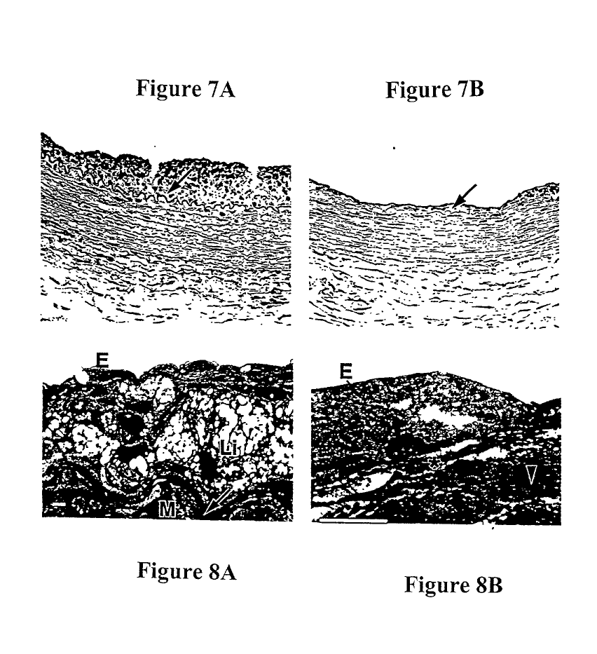

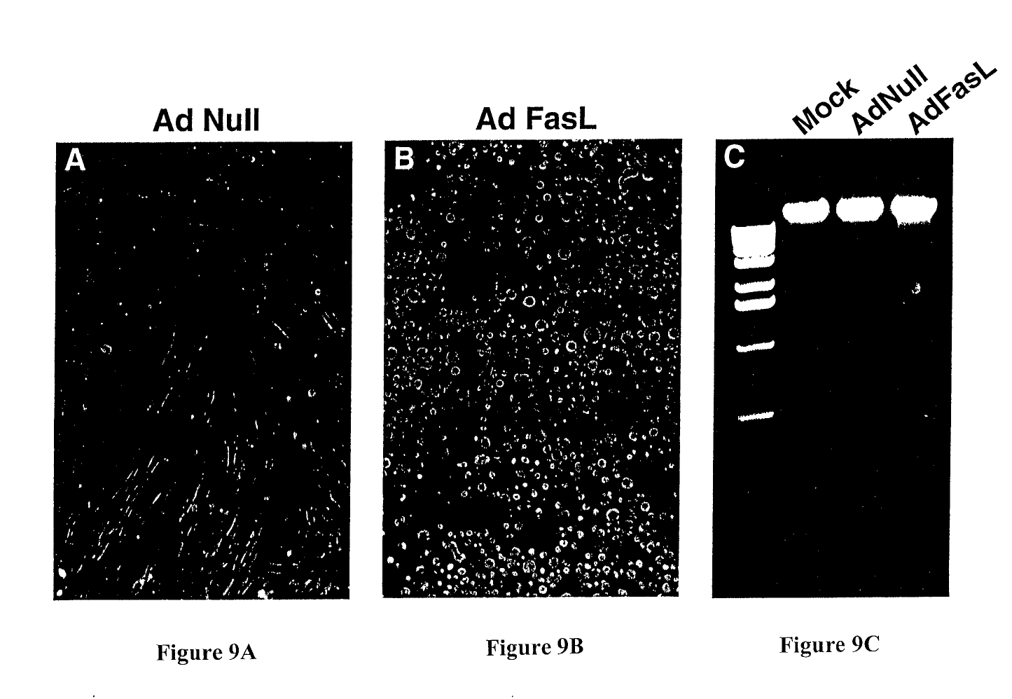

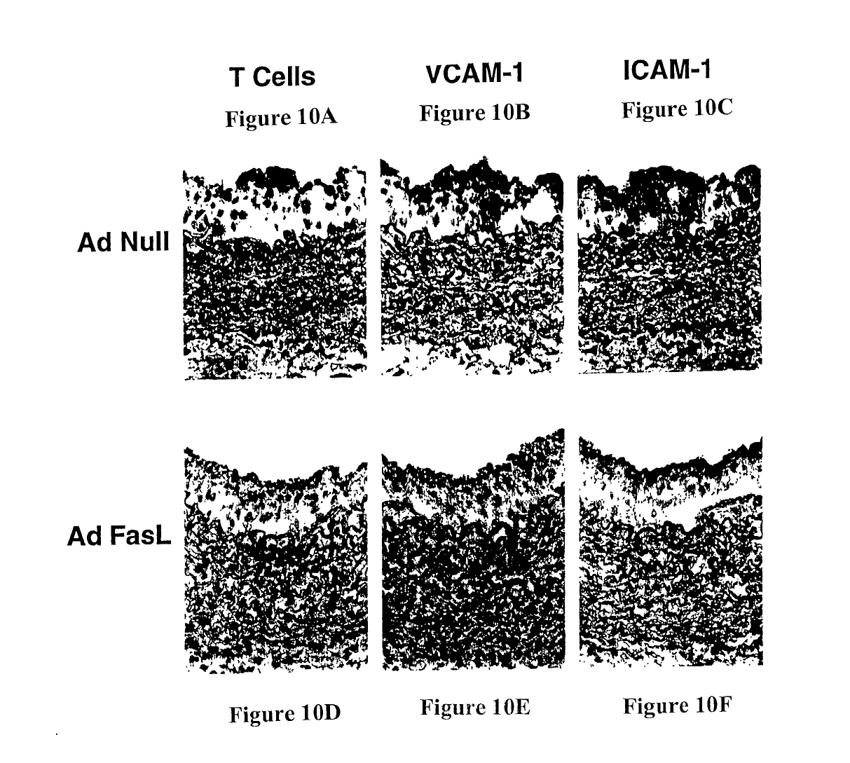

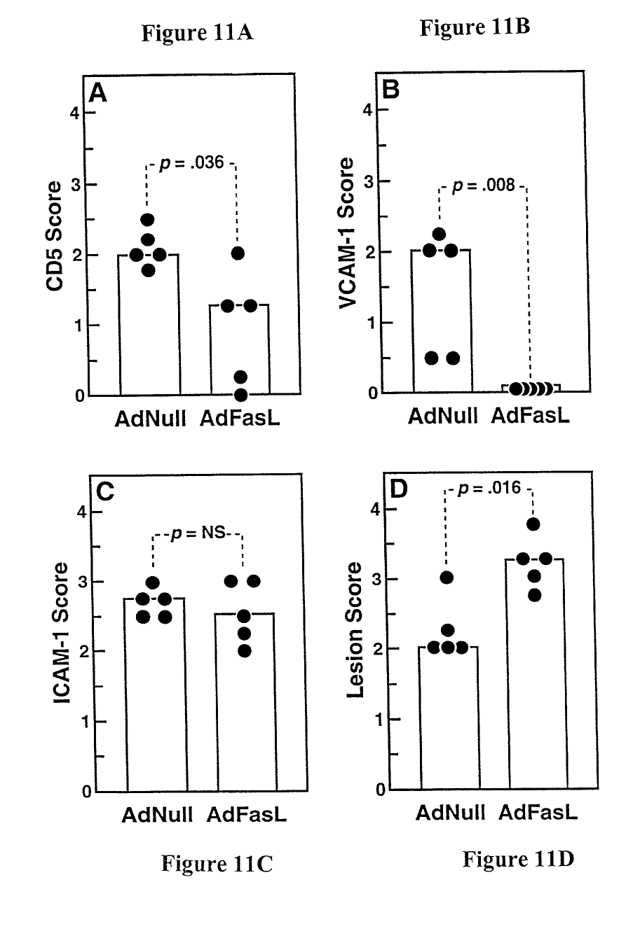

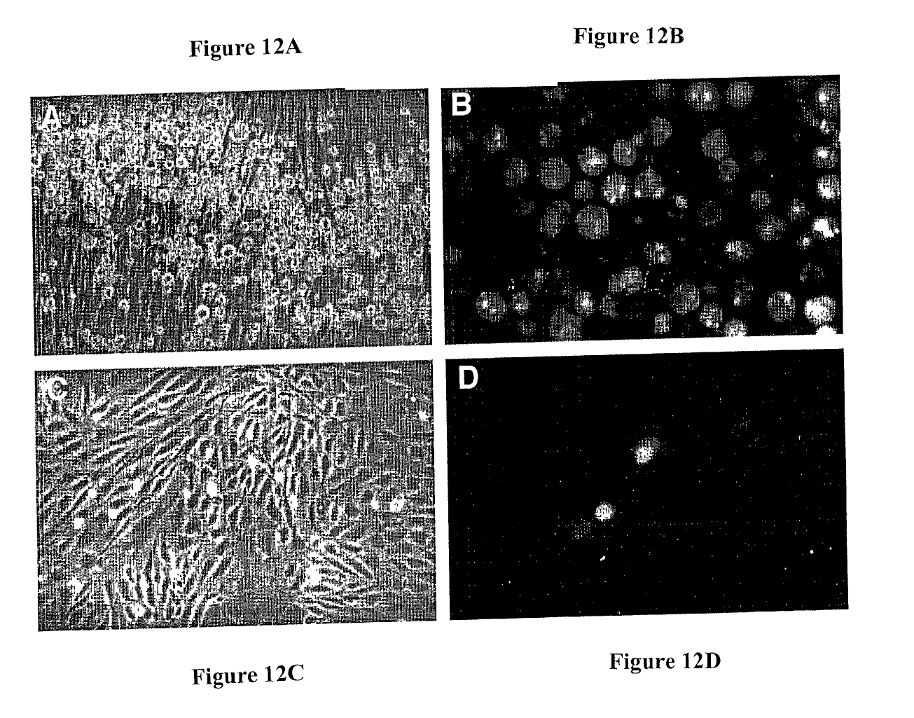

The resulting morphologies were distinct for each of the four depicted experimental models: (1) rabbit fed standard (normal) diet not sufficient to produce hypercholesterolemia, isolated rabbit carotid artery infused with vehicle solution without adenovirus (FIG. 2A); (2) rabbit fed standard (normal) diet not sufficient to produce hypercholesterolemia, isolated rabbit carotid artery infused with AdNull (FIG. 2C); (3) rabbit fed an atherogenic diet (e.g., high-cholesterol or high-fat diet) sufficient to produce hypercholesterolemia, isolated rabbit carotid artery infused with vehicle solution without adenovirus (FIG. 2B); (4) rabbit fed an atherogenic diet sufficient to produce hypercholesterolemia, isolated rabbit carotid artery infused with AdNull (FIG. 2D). These four experimental models and the stained arterial sections isolated therefrom were produced in accordance with the methods of the present invention, as described in greater detail below. Of these four models, the lesions produced by the infusion of the viral vector into the carotid artery of hypercholesterolemia-induced rabbits (FIG. 2D) most closely resembled lesions typically observed in early-stage human atherosclerotic lesions (having, e.g., intact endothelium, intima comprising macrophages and smooth muscle cells, lipid deposition similar to that typically early-stage human atherosclerotic lesions). Original magnifications are ×100 for each of FIGS. 2A-2D. A black arrow indicates the internal elastic lamina in each of FIGS. 2A-2D. The effects of hypercholesterolemia and adenovirus infusion into the carotid arteries are additive; that is, a larger lesion is produced with both hypercholesterolemia and adenovirus than with either alone.

FIGS. 2E-2H are microscopic views of sections of rabbit carotid arteries which show macrophage stains of the lesions of the four experimental animal models shown, respectively, in FIGS. 2A-2D. FIGS. 2E-2H were stained with RAM-11 antibody (which detects macrophages) and were counterstained with hematoxylin counterstain. Quantitative analysis of intimal mass and macrophage content of arteries harvested 4 weeks after infusion. Magnification was ×100 for FIGS. 2E-2H. A black arrow indicates the internal elastic lamina in each of FIGS. 2A-2D. FIGS. 2E-2H illustrate the macrophage content of carotid arteries from either normocholesterolemic rabbits or hypercholesterolemic rabbits that were infused with either vehicle solution or with a viral vector solution. FIG. 2E shows a section of an artery from a normocholesterolemic rabbit infused with vehicle solution; there is no intimal lesion and there are no macrophages present. FIG. 2F shows a section of an artery from a hypercholesterolemic rabbit infused with vehicle; there is an intimal lesion (above the internal elastic lamina) that contains a small number of macrophages (evidenced by the brown peroxidase reaction product). FIG. 2G shows a section of an artery from a normocholesterolemic rabbit infused with adenovirus. There is an intimal lesion (above the internal elastic lamina), but there are no macrophages present. FIG. 2H shows a section of an artery from a hypercholesterolemic rabbit infused with adenovirus. There is an intimal lesion (above the elastic lamina) and a large number of macrophages present (evidenced by the brown peroxidase reaction product).

FIGS. 3A-3B are graphical illustrations depicting a quantitative analysis of atherosclerotic lesions in rabbit carotid arteries. These data are from arterial lesions harvested from rabbits four weeks after such rabbits were subjected to the four above-identified experimental conditions. Methods for inducing and identifying lesions were performed according to the methods of the invention, as described herein and in more detail below. Data in FIGS. 3A and 3B are from the same arteries. FIG. 3A shows intimal-medial (I:M or I/M) area ratios for arteries from rabbits fed either standard (normal) diet or atherogenic diet (e.g., high-cholesterol or high-fat diet) and infused either with the AdNull adenovirus (+) or with vehicle solution only (−). Specifically, FIG. 3A shows the intimal:medial (I:M or I/M) ratios in rabbit carotid arteries harvested from rabbits of the following four experimental model groups: (1) rabbit carotid isolated from rabbit fed standard diet not sufficient to produce hypercholesterolemia was infused with vehicle solution without adenovirus and without cholesterol (−−); (2) rabbit carotid artery isolated from rabbit fed diet sufficient to produce hypercholesterolemia was infused with vehicle solution without adenovirus (−+); (3) rabbit carotid artery isolated from rabbit fed standard (normal) diet not sufficient to produce hypercholesterolemia was infused with adenovirus (+−) (AdNull); and (4) rabbit carotid artery isolated from rabbit fed diet sufficient to produce hypercholesterolemia was infused with adenovirus (++) (AdNull). The heights of the bars represent mean values calculated from the I:M ratios of the individual vessels in each group; error bars indicate standard error. Adenovirus infusion causes an inflammatory response in both the presence and absence of hypercholesterolemia.

FIG. 3B depicts the macrophage content of lesions produced in rabbit carotid arteries of the same four experimental models shown in FIG. 3A. The percentage of lesion area occupied by macrophages is quantitated by immunostaining using a macrophage-specific antibody to RAM-11 and computer-assisted image analysis in which the computer calculates the percentage of intimal area that contains the brown (peroxidase) staining color (i.e., %RAM-11 positive area per lesion). The heights of the bars represent mean values calculated from the %RAM-11 positive area per lesion for individual vessels in each group; error bars indicate standard errors. Macrophage accumulation was greatest in the lesion produced by the combination of virus infusion and induced hypercholesterolemia (FIG. 3B); this lesion most resembled a human atherosclerotic lesion. This is determined by the increased intimal area containing SMC and the observation of more RAM-11 staining due to the presence of more macrophages.

FIGS. 4A-4D are graphical illustrations depicting semiquantitative analyses of inflammation, vascular cell activation, and lesion size performed on stained sections of arteries harvested from rabbits subject to the following four experimental conditions: (1) rabbit carotid isolated from rabbit fed standard diet not sufficient to produce hypercholesterolemia was infused with vehicle solution without adenovirus (−−); (2) rabbit carotid artery isolated from rabbit fed diet sufficient to produce hypercholesterolemia was infused with vehicle solution without adenovirus (−+); (3) rabbit carotid artery isolated from rabbit fed standard diet not sufficient to produce hypercholesterolemia was infused with adenovirus (+−) (AdNull); and (4) rabbit carotid artery isolated from rabbit fed diet sufficient to produce hypercholesterolemia was infused with adenovirus (++) (AdNull). Methods for inducing and identifying lesions were performed according to the methods of the invention, as described herein and in greater detail below. The intensity of histochemical staining for detection of ICAM-1 expression, VCAM-1 expression, T cell infiltration as reflected in the CD5 score, and lesion size are shown. Antibody activity in each section was scored with the aid of a semiquantitative scale of staining intensity of 0 to 4, as described in Newman et al., J. Clin. Invest. 96:2955-2965 (1995): 0=no staining; 1=rare positive cells or staining barely visible at low (×100) magnification power; 2=focal staining or faint diffuse staining clearly visible at low (×100) magnification power; 3=multifocal staining or moderate intensity diffuse staining; 4=intense, diffuse staining. Neointimal lesions are scored by the same observers using a different semiquantitative scale: 0=no lesion; 1=partial circumference and less than 3 cells thick; 2=partial circumference and greater than 3 cells thick; 3=circumferential lesion. Four sections per vessel are scored by each of the two observers and the mean of these eight scores is used to generate a score for the entire vessel. The presence of inflammation in the lesions was dependent on infusion of adenovirus. Each y-axis represents the lesion score, using the semiquantitive scales of in vivo activity of the abundance of each antigen (FIGS. 4A-4C) or lesion (FIG. 4D). Each triangle represents one artery. Each data point represents mean values for a single artery. The heights of the bars represent median values calculated from the values of the individual vessels in each group.

FIG. 5 is a graphical illustration of the intimal:medial ratios of rabbit carotid arteries analyzed 7, 14, and 28 days, respectively, after infusion into the arteries of either control vector AdNull or an adenoviral vector containing Fas ligand (AdFasL). The number of arteries in each experimental group is shown (“n” values). The heights of the bars represent mean values calculated from the I:M ratios of the individual vessels in each group; error bars indicate standard errors. The effect of Fas ligand expression on atherosclerotic lesion development is demonstrated. Intimal lesion formation was significantly accelerated by infusion of AdFasL. At 7 and 14 days, the Intima:Media ratios in AdFasL-transduced arteries were greater than in AdNull-transduced arteries. These results suggest that activation of the FasL signalling pathway may contribute to atherosclerotic lesion development. Methods for inducing and identifying lesions, analyzing intimal:medial ratios, and gene transfer were performed according to the methods of the invention, as described herein and in greater detail below.

FIG. 6 is a graphical illustration of the percentage of lesion area in tissue sections occupied by macrophages, quantitated by using a monoclonal antibody specific for macrophages (i.e., RAM-11), infused with AdNull or AdFasL. The number of days after infusion on which the vessels were harvested is shown (7, 14, and 28 days, respectively). The number of arteries in each experimental group is indicated by the respective “n” value. The heights of the bars represent mean values calculated from the %RAM-11 positive area per lesion of the individual vessels in each group; error bars indicate standard errors. The lack of an effect of Fas ligand expression on the macrophage content of atherosclerotic lesions is demonstrated.

FIG. 7A is a photograph of an atherosclerotic lesion developing below intact endothelium of a carotid artery harvested from a hypercholesterolemic rabbit one week after infusion of AdFasL. An intimal lesion is present between the internal elastic lamina (arrow) and the endothelium. The endothelium is stained brown with peroxidase reaction product. FIG. 7B is a photograph of an atherosclerotic lesion developing below intact endothelium of a carotid artery harvested from a hypercholesterolemic rabbit one week after infusion of AdNull. A much smaller intimal lesion is present below the intact endothelium (brown) and the internal elastic lamina (arrow).

FIGS. 8A and 8B are transmission electron micrographs depicting the histology and ultrastructure of an induced experimental intimal atherosclerotic lesions in rabbit carotid arteries seven days after transduction. Sections are from arteries transduced with AdFasL (FIGS. 8A and 8B). FIG. 8A shows luminal endothelium (E), intimal lipid deposition (Li), a medial SMC with a “contractile” appearance (M). FIG. 8B is a higher magnification and shows rough endoplasmic reticulum (arrowhead), and extracellular collagen fibrils (asterisk). Size bars (lower left of FIGS. 8A and 8B) indicate 2 microns. Arrows indicate internal elastic lamina. Large amounts of lipid and phenotypically modified smooth muscle cells are seen below the intact endothelium.

FIG. 9A is a phase contrast micrograph of cultured rabbit smooth muscle cells that were exposed to AdNull (an adenoviral vector that does not express a transgene) at a concentration of 5×10⁹ particles/milliliter (ml). The original magnification of the micrograph is 200×. Note that cell viability is excellent. FIG. 9B is a phase contrast micrograph of cultured rabbit smooth muscle cells that were exposed to AdFasL (an adenoviral vector that expresses Fas ligand (FasL)) at a concentration of 5×10⁹ particles/ml. The original magnification of the micrograph is 200×. Note that cell viability is poor, and most of the cells are pyknotic and detached from the culture dish. FIG. 9C is a photograph of an agarose gel on which DNA extracted from smooth muscle cells (SMC) that were mock transduced, transduced with AdNull, or transduced with AdFasL was separated by electrophoresis. The gel was stained with ethidium bromide and DNA was visualized by illumination of the gel with ultraviolet light. DNA size markers (DNA from øX-174 RF digested with HaeIII) were also electrophesed and are present at the left of the gel. Fragmented DNA, typically found in cells that have undergone apoptosis, is found only in cells transduced with AdFasL.

FIGS. 10A-10F are photomicrographs of sections from rabbit carotid arteries that were transduced with either AdNull (FIGS. 10A-10C) or AdFasL (FIGS. 10D-10F). The arteries were removed from the rabbits 14 days after transduction, frozen, embedded in optimal cutting temperature medium, sectioned, and stained with antibodies that detect either T cells (FIGS. 10A and 10D), expression of vascular cell adhesion molecule 1 (VCAM-1; FIGS. 10B and 10E) or intercellular adhesion molecule 1 (1CAM-1; FIGS. 10C and 10F). Sections were counterstained with hematoxylin and photographed at an original magnification of 100×. Detailed methods were as described in Newman et al., Journal of Clinical Investigation 96:2955-2965 (1995). The brown peroxidase reaction product reveals bound antibody. Thus, in comparison to the artery transduced with AdNull (FIGS. 10A-10C), there is decreased presence of T cells and decreased expression of VCAM-1 and 1CAM-1 in the artery transduced with AdFasL.

FIGS. 11A-11D show graphical illustrations depicting a quantitative analysis of inflammation and lesion size in rabbit carotid arteries transduced with either AdNull or AdFasL and harvested 14 days later. Sections of arteries were stained with antibodies to CD5 (a T cell-specific antigen; FIG. 11A), vascular cell adhesion molecule 1 (VCAM-1; FIG. 11B) or intercellular adhesion molecule 1 (1CAM-1; FIG. 11C). Lesion size was graded on hematoxylin and eosin-stained sections (FIG. 11D). Intensity of antibody staining (FIGS. 11A-11C) and lesion size (FIG. 11D) were scored according to semiquantitative scales as described in detail below in the Detailed Description of the invention. Each data point represents a single artery. The heights of bars indicate the group medians.

FIGS. 12A-12D are photomicrographs of rabbit vascular cells transduced in vitro with AdFasL. Rabbit smooth muscle cells (FIGS. 12A-12B) and endothelial cells (FIGS. 12C-12D) were transduced with AdFasL at 3×10⁹ particles/milliliter (ml). Eighteen hours after exposure to AdFasL, cells were examined by phase contrast microscopy and photographed (FIGS. 12A-12C). Alternatively, the transduced cells were stained with “Yo-Pro-1”, a fluorescent, DNA-binding dye that is excluded from viable cells and then photographed by fluorescence microscopy (FIGS. 12B-12D). FIGS. 12C and 12D are not the same fields as FIGS. 12A and 12B. Original magnifications: FIGS. 12A and 12C are 51×; FIGS. 12B and 12D are 128×. Rabbit smooth muscle cells undergo apoptosis after transduction with AdFasL, as indicated by the pyknotic cells in FIG. 12A and the fragmented DNA in FIG. 12B. Rabbit endothelial cells are resistant to apoptosis induced by AdFasL, as indicated by the healthy appearance of cells in FIG. 12C and the absence of fragmented DNA in FIG. 12D.

### DETAILED DESCRIPTION OF THE INVENTION

I. Definitions

Unless defined otherwise, all technical and scientific terms used herein have the same meaning as commonly understood by those of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). As used herein, the following terms and phrases have the meanings ascribed to them unless specified otherwise. Although any methods and materials similar or equivalent to those described herein may be used in the practice or testing of the present invention, the preferred methods and materials are described. For purposes of the present invention, the following terms and phrases are intended to have the following general meanings as they are used herein:

The term “atherogenic diet” as used herein means a diet of food, drink, and/or other compounds and substances (e.g., cholesterol-rich or high-cholesterol diet) that has the capacity to initiate, increase, induce, produce, accelerate, or cause a risk of a characteristic, symptom, or condition associated with or related to atherogenesis and/or arteriosclerosis. An atherogenic diet may be one that comprises an amount of cholesterol that is in excess of that recommended for a standard or normal diet. In one particular aspect, an atherogenic diet may comprise an amount of cholesterol having a capacity to initiate, increase, induce, produce, accelerate, or cause a risk of a characteristic, symptom, or condition associated with or related to atherogenesis and/or arteriosclerosis in a subject. Such an amount of cholesterol depends on the subject (e.g., non-human mammal) to which it is administered and the conditions under which it is administered. For example, large animals may require a greater amount of cholesterol than small animals to effectuate the above-described capacity. Animals at risk of atherosclerosis or already presenting signs of such disease may require less than animals not at risk of or manifesting signs of the disease. Further, the metabolism of some animals may dictate or necessitate a specific amount of cholesterol to produce the above-described capacity.

An atherogenic diet administered to a rabbit or similar non-human mammal, for example, may comprise, among other things, administration of an amount of from about, as a percentage of the total dietary intake, 0.05% to about 2% cholesterol, or from about 0.05% to about 0.15% cholesterol, or from about 0.15% to about 0.25% cholesterol, or from about 0.25% to about 1% cholesterol, or from about 1% to about 2% cholesterol. In addition, an atherogenic diet may include an amount of lipids, fats, triglycerides, fatty acids, and related compounds (e.g., soybean oil, linoleic acid, oleic acid, palmitic acid, stearic acid, etc.), the presence of which contributes to the formation of arterial lesions. Examples of such lipids, fats, triglycerides, fatty acids, and other related dietary substances that contribute to atherogenesis in animal models are found in T. Clarkson, Advances in Lipid Research 1:221-252 (1963) and E. Wissler, Ann. N.Y. Acad. Sci. 149:907 (1968), both of which references are incorporated herein in their entirety for all purposes; such atherogenic diets may comprise the administration to a subject or similar non-human mammal of an amount of such a lipid, fat, triglyceride, fatty acid, or related compound in an amount ranging from about 1% to about 20% as a percentage of the total dietary intake as specified above for cholesterol.

The term “hypercholesterolemic diet” means a diet of food, drink, and/or other compounds and substances (e.g., cholesterol) which comprises, among other things, an amount of cholesterol sufficient to induce or produce a condition of hypercholesterolemia in the subject to which the diet is administered. Hypercholesterolemia is a condition characterized by the presence of an abnormally large amount of cholesterol in the cells and the plasma of the circulating blood. Such an amount of cholesterol depends on the subject (e.g., non-human mammal) to which it is administered and the conditions under which it is administered. For example, large animals may require a greater amount of cholesterol than small animals to induce or produce hypercholesterolemia. Animals may be genetically inclined to hypercholesterolemia or hyperlipidemia and, as a result, may present signs of such diseases or conditions without having ingested an amount of cholesterol in excess of that present in a standard or normal diet. Animals at risk of hypercholesterolemia or already presenting signs of such disease may require less than animals not at risk of or manifesting signs of the disease. Further, the metabolism of some animals may dictate or necessitate a specific amount of cholesterol to produce hypercholesterolemia. For a rabbit, a hypercholesterolemic diet may comprise, among other things, e.g., as a percentage of the total dietary intake, administration of from about 0.05% to about 2% cholesterol, or from about 0.05% to about 0.15% cholesterol, or from about 0.15% to about 0.25% cholesterol, or from about 0.25% to about 1% cholesterol, or from about 1% to about 2% cholesterol. A hypercholesterolemic diet may include an amount of lipids, fats, triglycerides, fatty acids, and related compounds (e.g., soybean oil, linoleic acid, oleic acid, palmitic acid, stearic acid, etc.), the presence of which contributes to the formation of arterial lesions. Examples of such lipids, fats, triglycerides, fatty acids, and other related dietary substances that contribute to atherogenesis in animal models are found in Clarkson, supra, and Wissler, supra; such hypercholesterolemic diets may comprise the administration to a subject or similar non-human mammal of an amount of such a lipid, fat, triglyceride, fatty acid, or related compound in an amount ranging from about 1% to about 20% as a percentage of the total dietary intake as specified above for cholesterol. Rabbit feeding of the amounts of cholesterol, lipids, fats, triglycerides, fatty acids, or other related dietary substances necessarily produces hypercholesterolemia. Hypercholesterolemia is defined as two (2) standard deviations above mean for cholesterol for animals eating a standard, normal diet.

The term “cholesterol-rich diet” as used herein means a diet of food, drink, and/or other compounds and substances which comprises, among other things, an amount of cholesterol sufficient to induce or produce hypercholesteremia or hyperlipidemia in a subject to which it is administered. Such an amount of cholesterol depends on the subject to which it is administered and the conditions under which it is administered. For example, large animals may require a greater amount of cholesterol than small animals to induce or produce hypercholesteremia or hyperlipidemia. Animals at risk of hypercholesterolemia or hyperlipidemia or already presenting signs of such diseases may require a lesser amount of cholesterol than animals not at risk of or manifesting signs of such diseases. Animals may be genetically inclined to hypercholesterolemia or hyperlipidemia and, as a result, may present signs of such diseases or conditions without having ingested an amount of cholesterol in excess of that present in a standard or normal diet. Further, the metabolism of some animals may dictate or necessitate a specific amount of cholesterol to produce hypercholesterolemia or hyperlipidemia. For a rabbit, a cholesterol-rich diet may comprise, among other things, e.g., as a percentage of the total dietary intake, administration of from about 0.05% to about 2% cholesterol, or from about 0.05% to about 0.15% cholesterol, or from about 0.15% to about 0.25% cholesterol, or from about 0.25% to about 1% cholesterol, or from about 1% to about 2% cholesterol. A cholesterol-rich diet may include an amount of lipids, fats, triglycerides, fatty acids, and related compounds (e.g., soybean oil, linoleic acid, oleic acid, palmitic acid, stearic acid, etc.), the presence of which contributes to the formation of arterial lesions. Examples of such lipids, fats, triglycerides, fatty acids, and other related dietary substances that contribute to atherogenesis in animal models are found in Clarkson, supra, and Wissler, supra; such cholesterol-rich diets comprise the administration to a subject or similar non-human mammal of an amount of such a lipid, fat, triglyceride, fatty acid, or related compound in an amount ranging from about 1% to about 20% as a percentage of the total dietary intake as specified above for cholesterol.

The term “standard diet” or “normal diet” means a diet of food, drink, and/or other compounds and substances which comprises, among other things, either no cholesterol or an amount of cholesterol that is not sufficient to produce a condition of hypercholesterolemia or atherosclerosis in the subject to which the diet is administered. A standard (normal) diet consisted of High Fiber Laboratory Rabbit Diet #5326 (Dean's Feed, Belmont, Calif.).

The term “atherosclerotic lesion” means a lesion or pathological alteration of the tissue of a blood vessel (e.g., artery or vein) or its tissue. Atherosclerosis is manifested by a variety of different lesions. The earliest lesions of atherosclerosis are usually found in young subjects (e.g., infants) in the form of a lesion called the “fatty streak.” See E. Braunwald, HEART DISEASE: A TEXTBOOK OF CARDIOVASCULAR MEDICINE 1140-1142 (3d. ed 1988), which is incorporated herein by reference in its entirety for all purposes. The fatty streak consists of, among other things, lipid-laden macrophages, with varying amounts of lipid-filled smooth muscle cells that accumulate beneath them as the lesions grow in size. Id. at 1141. Most of the lipid is cholesterol and cholesteryl ester. Id. Foam cells form in the fatty streaks in advanced lesions. Id. The advanced lesion, termed the “fibrous plaque,” typically appears during early development of the subject (e.g., childhood) and progresses with age. Id. at 1140. These lesions consist of large numbers of intimal smooth muscle cells, along with many macrophages. Id. at 1141. When these lesions hold lipid, such lipid is typically cholesterol or cholesteryl ester. Id. at 1141-1142. The proliferating smooth muscle cells are surrounded by collagen and elastic fibers, by significant amounts of proteoglycans, and in subjects who are hypercholesterolemic, by differing amounts of lipid deposited in the cells and connective tissue. Id. at 1142.

Another type of atherosclerotic lesion is a diffuse intimal thickening which consists of increased numbers of intimal smooth muscle cells surrounded by variable amounts of connective tissue. Id. at 1141. Characteristics of the different atherosclerotic lesions and their respective development are described in detail in Braunwald, supra.

The phrase “atherosclerotic lesion development” refers to the development or occurrence of an atherosclerotic lesion.

The phrase “activity on atherosclerotic lesion development” is intended to mean an action or operation on or change in the development of an atherosclerotic lesion in a vascular element of a subject (e.g., blood vessel of a non-human mammal), including an increase or decrease in atherosclerotic lesion development of the subject, as indicated or manifested by, for example, a property (as defined in detail below), such as a clinical manifestation, characteristic, symptom, or event that occurs in or is observed in, associated with, or peculiar to an element of the vascular system of the subject (e.g., blood vessel, artery or vein) that is affected by, involved in, at risk of, subject to, associated with, or manifesting atherosclerosis and/or atherosclerotic lesion development. Characteristics, clinical manifestations, and symptoms of atherosclerotic lesion development in blood vessels are described in E. Braunwald, HEART DISEASE: A TEXTBOOK OF CARDIOVASCULAR MEDICINE, Chap. 35 (3d ed. 1988).

The phrase “anti-atherosclerotic activity” is intended to mean an action or operation on or change in the development of an atherosclerotic lesion in a vascular element of a subject (e.g., blood vessel of a non-human mammal), which is a manifest by a decrease in atherosclerotic lesion development of the subject.

The phrase “property of the blood vessel to indicate an activity on atherosclerotic lesion development” is intended to mean a clinical manifestation, characteristic, symptom, or event that occurs in or is observed in, associated with, or peculiar to an element of the vascular system (e.g., a blood vessel, such as an artery or vein) of a subject (including a non-human mammal) that is affected by, involved in, at risk of, subject to, associated with, or manifesting atherosclerosis and/or atherosclerotic lesion development. A property of the vascular element (e.g., artery or vein) indicating atherosclerosis and/or atherosclerotic lesion development is monitored to ascertain whether atherosclerotic lesion development is occurring, decreasing, increasing, being prevented, etc. Properties of vascular elements or blood vessels, such as arteries or veins, that are monitored for such atherosclerotic lesion development include, among other things, proliferation or accumulation of smooth muscle cells, inflammatory T cells, and macrophages in the blood vessel; increase in the volume of the intima of the blood vessel; narrowing of the lumen of the blood vessel; decrease in blood flow through the blood vessel; accumulation of lipid in the wall of the vessel; accumulation of extracellular matrix in the wall of the vessel; and rupture of a plaque in the blood vessel. Additional characteristics, clinical manifestations, or symptoms of such lesion development are described in E. Braunwald, supra, Chap. 35 (3d ed. 1988). The blood vessel whose property is monitored can comprise an artery or vein. An “arterial property” is such a property of an artery; a “venous property” is such a property of a vein.

The term “effective amount” means a dosage sufficient to produce a desired result. The desired result may comprise a subjective or objective improvement in the recipient of the dosage.

A “prophylactic treatment” is a treatment administered to a subject who does not exhibit signs or symptoms of a disease or exhibits only early signs or symptoms of a disease, wherein treatment is administered for the purpose of diminishing, decreasing, or preventing the risk of developing pathology. A prophylactic treatment acts as a preventive against a disease. A “prophylactic activity” is an activity of e.g., an agent, gene, nucleic acid segment, pharmaceutical, substance, compound, or composition which, when administered to a subject who does not exhibit signs or symptoms of a disease or exhibits only early signs or symptoms of a disease, diminishes, decreases, or prevents the risk in the subject of developing pathology. The term “prophylactically useful” in reference to a compound or agent (including, e.g., a protein or polypeptide) means that such compound or agent is useful is diminishing, decreasing, or preventing a pathology or disease.

A “therapeutic treatment” is a treatment administered to a subject who exhibits signs or symptoms of pathology, wherein treatment is administered for the purpose of diminishing or eliminating those pathological signs or symptoms. A “therapeutic activity” is an activity of e.g., an agent, gene, nucleic acid segment, pharmaceutical, substance, compound, or composition, which diminishes or eliminates pathological signs or symptoms when administered to a subject exhibiting the pathology. The term “therapeutically useful” in reference to a compound or agent (including, e.g., a protein or polypeptide) means that such compound or agent is useful in diminishing, decreasing, treating, or eliminating pathological signs or symptoms of a pathology or disease.

The term “non-human mammal” is intended to mean a mammal which is not a human, including, for example, a rabbit, mouse, pig, non-human primate (e.g., monkey) or other mammal. A non-human mammal subject to treatment and for use in methods of the present invention includes, for example, a rabbit, mouse, pig, non-human primate (e.g., monkey), or other mammal.

The term “non-human mammal model” is intended to mean a mammalian model which is not a human, including, for example, a rabbit, mouse, pig, non-human primate (e.g., monkey), or other non-human mammal.

The term “subject” is intended to mean an animal, such as a mammal, including a human. A non-human subject for use according to methods of the present invention includes, for example, a rabbit, mouse, pig, non-human primate (e.g., monkey), or other non-human mammal.

The term “test agent” is intended to mean an agent which is being tested for an activity on atherosclerosis, atherosclerotic lesion development, and/or atherosclerotic disease conditions. Such a test agent includes a nucleic acid segment, which may include a gene that has prophylactic or therapeutic effects (or negative effects), a vector, including a non-viral or viral vector, a pharmaceutical or pharmacologic compound or drug, toxin, natural product, or chemical compound. In some gene therapy methods, a test agent may comprise a nucleic acid segment containing a particular gene that is being screened for its activity or effect on atherosclerosis, atherosclerotic lesion development, and/or atherosclerotic disease conditions. In one aspect, such nucleic acid segment may consist of the gene being screened, without unnecessary additional nucleotides.

The term “natural product” is intended to include an organic molecule isolated or purified from a plant, animal, yeast or bacterium. A natural product includes, e.g., among other things, organic molecules belonging to the broad biochemical classes of proteins, carbohydrates, and lipids, as well as more complex molecules which comprise, e.g., elements of more than one of these basic biochemical classes.

The term “proinflammatory agent” is intended to mean an agent that induces, causes, or stimulates an inflammatory response or effect in a tissue or vascular element (e.g., artery or vein) of a subject or non-human mammal to which it is administered or delivered or with which it is contacted. An inflammatory response or effect is one which is made apparent or manifest by or is associated with neointimal hyperplasia, inflammation, and vascular cell activation and includes proliferation and/or accumulation of inflammatory T cells, macrophages, and smooth muscle cells, expression of adhesion molecules, secretion of cytokines or chemokines, increases in vascular permeability. A proinflammatory agent includes, for example, a vector, nucleic acid segment, cytokine, toxin, chemokine, or chemical compound. The proinflammatory agent may act directly or indirectly on the blood vessel (e.g., artery or vein). Such vector may be a viral or non-viral vector.

An exogenous DNA segment is one foreign (or heterologous) to the cell or homologous to the cell but in a position within the host cell nucleic acid in which the element is not ordinarily found. Exogenous DNA segments are expressed to yield exogenous polypeptides or RNA with a specific biological activity, e.g., a ribozyme.

The term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins.

The term “nucleic acid” or “nucleic acid segment” refers to a deoxyribonucleotide or ribonucleotide and polymer thereof which is in either single- or double-stranded form. Unless specifically limited, the term encompasses nucleic acids containing known analogues (synthetic and naturally occurring) of nucleotides, which have similar binding properties as the reference nucleic acid and are metabolized in a manner similar to the reference nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, and mRNA encoded by a gene.

The term “isolated nucleic acid” or “isolated nucleic acid segment” means a nucleic acid (e.g., an RNA, DNA, or a mixed polymer), which is substantially separated from other genome DNA sequences as well as proteins or complexes such as ribosomes and polymerases, which naturally accompany a native sequence. The term embraces a nucleic acid sequence which has been removed from its naturally occurring environment, and includes recombinant or cloned DNA isolates and chemically synthesized analogues or analogues biologically synthesized by heterologous systems. A substantially pure molecule includes isolated forms of the molecule. An “isolated polypeptide” or protein carries a similar meaning with the polypeptide or protein being substantially separated from any cellular contaminants and components naturally associated with the protein in vivo.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under expressed or not expressed at all. The term “recombinant DNA molecule,” for example, refers to a nucleic acid sequence which is not naturally occurring, or is made by the artificial combination of two otherwise separated segments of sequence. By “recombinantly produced” is meant artificial combination often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. “Recombinant DNA molecules” include cloning and expression vectors.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “a sequence encoding a gene product” refers to a nucleic acid that contains sequence information, e.g., for a structural RNA such as rRNA, a tRNA, the primary amino acid sequence of a specific protein or peptide, a binding site for a trans-acting regulatory agent, an antisense RNA or a ribozyme. This phrase specifically encompasses degenerate codons (i.e., different codons which encode a single amino acid) of the native sequence or sequences which may be introduced to conform with codon preference in a specific host cell.

The technique of “polymerase chain reaction,” or “PCR,” as used herein generally refers to a procedure wherein minute amounts of a specific piece of nucleic acid, RNA and/or DNA, are amplified as described in U.S. Pat. No. 4,683,195, issued Jul. 28, 1987. Generally, sequence information from the ends of the region of interest or beyond needs to be available, such that oligonucleotide primers can be designed; these primers will be identical or similar in sequence to opposite strands on the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See, generally, Mullis et al., Cold Spring Harbor Symp. Quant. Biol., 51:263 (1987); PCR Technology, (Erlich, ed., Stockton Press, N.Y., 1989). As used herein, PCR is considered to be one, but not the only, example of a nucleic acid polymerase reaction method for amplifying a nucleic acid test sample, comprising the use of a known nucleic acid (DNA or RNA) as a primer.

The term “antibody” refers to a polypeptide substantially encoded by an immunoglobulin gene or fragments thereof that specifically binds and recognizes an antigen. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies or those synthesized de novo using recombinant DNA methodologies.

The term “immunoassay” is an assay that uses an antibody to specifically bind an antigen. The immunoassay is characterized by the use of specific binding properties of a particular antibody to isolate, target, and/or quantify the antigen.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “isolate” as used herein in reference to a vascular element or vessel (e.g., artery, arterial tissue, vein, etc.) refers to separating or setting apart such element from another element(s) or object(s), or to free from contaminants. A reference to isolating a vascular element (e.g., artery, arterial tissue, vein, etc.), as by the methods of the present invention described in detail below, means to separate, detach, disengage, segregate or set apart such element from another element(s) or objects(s) either in vivo or, alternatively, to separate, detach, disengage, segregate or set apart such element from a host subject (e.g., animal or human) such that it can be used in ex vivo or in vitro methods, including screening methods of the present invention. In some methods of the present invention described in detail below, a vascular element or blood vessel (e.g., artery or vein) can be surgically isolated by clamping a segment of the vessel between atraumatic vascular clamps to facilitate observation, testing, and analysis of such vessel and vessel segment. The term “surgically isolated” means isolation of the element or object by surgical means or procedures. A vascular element or vessel can also be isolated between two catheters inserted percutaneously, by using two balloons mounted on the same catheter or by using any other type of catheter device that is adapted to be useful for the local delivery of substances to the artery wall.

The term “toxin” means generally a poisonous substance, including, e.g., a biological compound (e.g., having a protein structure) or chemical compound or similar substance which is capable of causing a toxic response in a subject which comes into contact with it. Such a substance may be secreted by an organism and may be capable of causing toxicosis when introduced into body tissues. Such a substance may be capable of inducing a counteragent or an antitoxin.

Generally, the nomenclature used hereafter and the laboratory procedures in cell culture, molecular genetics, and nucleic acid chemistry described below are those well known and commonly employed in the art. Standard techniques such as described in Sambrook et al., Molecular Cloning, A Laboratory Manual, (Cold Spring Harbor Laboratory, Cold Spring Harbor, N.Y., 2nd ed. 1989), are used for recombinant nucleic acid methods, nucleic acid synthesis, cell culture, and transgene incorporation, e.g., electroporation, injection, lipofection. Generally, oligonucleotide synthesis, and purification steps are performed according to the specifications. The techniques and procedures are generally performed according to conventional methods in the art and various general references which are provided throughout this document. The procedures therein are believed to be well known in the art and are provided for the convenience of the reader.

II. Atherosclerosis: Development and Manifestations

Atherosclerosis is a multistage, multifactorial process which is stimulated when cells lining the blood vessels (e.g., arteries) are damaged or deteriorate as a result of high blood pressure, genetic factors, smoking, environmental toxins, mechanical or other injury, immunologic injury, viruses, chemicals, pharmaceuticals, and other agents. STEDMAN'S MEDICAL DICTIONARY 162 (Williams & Wilkins, 26th ed. 1995), which is incorporated herein by reference in its entirety for all purposes. Atherosclerosis involves considerable proliferation of smooth muscle cells within the intima of the affected vessel (e.g., artery). See Braunwald, supra, Chap. 35, pp. 1135-1152 (3d ed. 1988). Atherosclerosis is characterized by irregularly distributed lipid deposits in the intima of large and medium-sized blood vessels, such as the arteries. STEDMAN'S MEDICAL DICTIONARY, supra, at 162. Such deposits cause fibrosis and calcification. Id. Plaques develop in the arteries when high density lipoproteins accumulate at the site of arterial damage and platelets work to form a fibrous cap over this fatty core. See, e.g., Braunwald, supra. These deposits inhibit and can ultimately completely block the flow of blood. Id. Such plaques, which are more or less calcified depending on the progress of the disease, can be associated with lesions and are related to the accumulation in the arteries of fatty deposits that consist of, among other things, cholesteryl esters. In the arteries, a thickening of the artery wall and hypertrophy of smooth muscle occurs along with plaque formation. The disease leads to the reduction of the arterial lumen and predisposes to thrombosis. A similar process can also occur in vein segments that have been surgically connected to the arterial system.

Based on the morphology and content of advanced atherosclerotic lesions, it is believed that the disease is primarily the result of three biological processes: 1) the proliferation of intimal smooth muscle cells along with variable numbers of accumulated macrophages: 2) the formation of large amounts of connective tissue matrix, including collagen, elastic fibers, and proteoglycans, by the proliferated smooth muscle cells; and 3) the accumulation of lipids (e.g., particularly cholesteryl esters and free cholesterol) within the cells and the surrounding connective tissues. Braunwald, supra, at 1135.

The normal artery comprises an intima, media, and adventitia. In some species, such as the rabbit, mouse, and rat, the intima comprises a single layer of endothelium. In other species, the intima comprises also connective tissue with smooth muscle cells. In all species, the intima is lined by endothelium on the inner (luminal) aspect of the arterial vessel and by the internal elastic lamina on its outer aspect. Id. at 1135-1136. The media is the muscular wall of the artery and, in a normal artery, is bounded by the internal elastic lamina and an external elastic lamina in well-developed muscular and elastic arteries. Id.. The adventitia is a highly vascular tissue, which consists of a dense collagenous structure containing many bundles of collagen fibrils, elastic fibers and many fibroblasts with some smooth muscle cells, is bounded by the elastic lamina and the exterior of the vessel itself. Id. For a general review of the structure of the normal artery, see Braunwald, supra, Chap. 35 (3d ed. 1988).

Endothelial cells line the entire vascular system in a monolayer format. Id. at 1137. Endothelial cells form a continuous, smooth, uninterrupted surface in the vascular network and constitute the primary barrier between the elements of blood and the arterial wall. These cells control the passage of molecules and cells into the vessel. Id. at 1136. In addition, endothelial cells are capable of modulating tone and secreting factors that affect the growth properties of other vascular cells.

Smooth muscle cells in the artery help to preserve the tone of the arterial wall by their ability to maintain slow contractions. Id. Generally, for atherosclerotic lesions to form, smooth muscle cells must migrate from the media into the arterial intima. Id. at 1139. Advanced atherosclerotic lesions result, in part, from the proliferation of smooth muscle cells in the intima. The smooth muscle cells observed in atherosclerotic lesions are typically characterized by an accumulation of lipid that causes the formation of vacuolated cells or foam cells. Id. at 1139. Most lipids deposited in these smooth muscle cells consist of cholesteryl esters resulting from an increase in cholesterol synthesis and esterification and a decrease in degradation of cholesteryl esters in the lysosomes of the cell. Id. at 1139.

Macrophages are also typically present in the intima within human atherosclerotic lesions. A macrophage is a mononuclear, actively phagocytic cell arising from the monocytic stem cells in the bone marrow. STEDMAN'S MEDICAL DICTIONARY, supra, at 1051. Macrophages are dispersed throughout the body and vary in morphology and motility. Most macrophage cells are large and have numerous lysosomes, phagolysosomes, and endocytic vacuoles. Id. Macrophages are involved in the production of antibodies and in cell-mediated immune responses. Id. In general, macrophages serves as scavenger cells in inflammatory sites within the body, removing foreign substances by phagocytosis and intracellular hydrolysis. Braunwald, supra, at 1139. Macrophages secrete a vast number of biologically important substances, including chemotactic agents and oxygen metabolites. They also form and secrete growth factors. Id. at 1140. Macrophages are believed to be responsible for the promotion of connective tissue proliferation associated with chronic inflammatory responses. Id. Along with smooth muscle cells, macrophages are a principal source of the foam cells observed in human atherosclerotic lesions. Id. They are the primary cells in the initial atherosclerotic lesion—which is called the “fatty streak.” Id.

In atherosclerotic lesions in the blood vessels (e.g., arteries) of mammals, including humans, such vessels (e.g., arteries) are typically characterized as having a histologically intact endothelium and an intima comprising numerous intimal smooth muscle cells as well as T cells and macrophages. Atherosclerotic lesions in mammals typically comprise fibrous plaques which contain the intimal smooth muscle cells, macrophages, and T cells. Lipids and lipoproteins may be present in atherosclerotic (and hypercholesterolemic) lesions (including, e.g., in smooth muscle cells), as cholesterol or cholesteryl esters. See, e.g., Braunwald, supra, at 1145.

In some presently known animal models of atherosclerosis, intimal smooth muscle disease has been induced by passing an intra-arterial balloon embolectomy catheter through an artery sufficient to strip off the lining endothelium. Id. at 1139. The exposed subendothelial connective tissue attracts platelets to adhere and degranulate. Id. The smooth muscle cells migrate from the media into the intima, where they proliferate and form a myointimal, hyperplastic, fibrotic lesion. Id. In addition, human atherosclerosis is characterized by inflammation and accumulation of lipid in the blood vessel wall.

The clinical manifestations and characteristics of atherosclerosis are observed principally in medium-sized and large muscular arteries, including the carotid, coronary, and vertebral arteries. In addition, atherosclerosis is found in arteries of the lower extremities, such as the femoral and popliteal arteries. Id. The disease is also observed in larger arteries, such as aorta and iliac arteries. Id. Further, atherosclerosis is seen in brachial, subclavian, and innominate arteries.

Various types of cells in atherosclerotic lesions can be identified by a variety of techniques. Monoclonal antibodies against smooth muscle-alpha actin and against cytoplasmic antigen in macrophages, for example, are used to identify smooth muscle cells and macrophages, respectively, in atherosclerotic lesions. Methods for fixing and preserving tissues containing atherosclerotic lesions and using monoclonal antibodies specific for smooth muscle cells and macrophages are also well known to those of ordinary skill in the art. Tsukada et al., Am. J. Path. 126:51-60 (1987); Tsukada et al., Arteriosclerosis 6:601-613 (1986).

It is believed that atherosclerotic lesions and atherosclerosis can be induced by (non-denuding) injury to the lining of endothelial cells at certain sites in the arterial wall. Id. at 1142-1143. Injury can be induced by, for example, hypercholesterolemic or hyperlipidemic conditions. Id. at 1143. An increase in plasma lipoproteins (e.g., low-density lipoproteins, cholesterol) produces changes in the surface characteristics of endothelial cells and leukocytes (e.g., circulating monocytes and platelets) and an increase in adhesion of monocytes to endothelium throughout the arterial system. Id. Monocytes which adhere to the endothelium are believed to be chemotactically attracted to move between endothelial cells and to localize subendothelially. Such monocyte cells begin to act as scavengers and are converted to macrophages in the subendothelium. These cells enlarge and take up lipid. The lipid may enter the subendothelium in large amounts under hypercholesterolic conditions. Id. The accumulation of lipid in the artery wall cells results in the production of foam cells and fatty streaks. Id. at 1146-1147. An ongoing process of endothelial injury and macrophage buildup and stimulation, which produces activated endothelial cells and activated macrophages, contributes to the continued development of atherosclerotic lesions. Id. at 1144.

Atherosclerosis has been found to increase the risk of heart attack, stroke, myocardial infarction, angina, ischemia, and other forms of heart disease. Atherosclerosis has been associated with certain genetic features, obesity, high-fat diet, high blood pressure, hypertension, hypercholesteremia or hyperlipidemia, hormone dysfunction, diabetes, and cigarette smoking. Preventative measures include a low-fat diet (especially maintaining a diet low in cholesterol), regular exercise and control of obesity, control of high blood pressure, control of hyperlipidemia and hypercholesterolemia, control of hormone dysfunction, control of diabetes, and avoidance of tobacco.

The plasma of a human with atherosclerotic disease typically has a cholesterol levels of from about 180 to 300 milligram/deciliter (mg/dl).

III. Non-Human Mammalian Models of Atherosclerosis

Generally speaking, mice and rabbits are the most widely used animal models. Both murine and rabbit animal models are easy to manipulate and inexpensive. However, these small mammals are not always compatible with the intended application and methods or representative of the human model and its metabolic processes. The chimpanzee is a test animal, closer to man, which is used for screening for therapeutic and prophylactic agents directed to human diseases. The chimpanzee model, though, is very costly. Although the methods of the present invention can be readily practiced with a wide variety of mammals, we selected the rabbit as a non-human mammalian model for study, because it is especially useful as an animal model of vascular disease. Furthermore, we have comprehensive knowledge of the metabolism and diseases (including vascular diseases) of the rabbit.

Given the serious and prevalent nature of vascular diseases, it is useful to have available an animal model which is capable of expressing a protein or peptide which can prevent or treat vascular diseases and for screening test agents for their ability to therapeutically or prophylactically treat or prevent such diseases. Such an animal model is particularly advantageous for understanding vascular diseases and the regulatory mechanisms they initiate. With such an animal model, it is possible to test—rapidly and in vivo—a considerable number of therapeutic and/or prophylactic agents for the purpose of detecting a potential activity associated with a vascular disease (e.g., atherosclerosis and atherosclerotic lesion development). Additionally, such an animal model is useful for developing novel therapeutic and prophylactic methods of treating vascular diseases, including, e.g., methods based on gene therapy.

In one aspect, the present invention provides non-human mammalian models for atherosclerotic disease. Such models are useful for screening agents for therapeutic and prophylactic effects and activity on atherosclerosis and for use in gene transfer therapies for treating and preventing atherosclerosis. Such models comprise a non-human mammal characterized by having an atherosclerotic lesion in a blood vessel, wherein the blood vessel has a histologically intact endothelium and an intima comprising smooth muscle cells, and the lesion contains a proinflammatory agent. In some such models, the intima further comprises T cells or macrophages. The proinflammatory agent may comprise a wide variety of compounds or substances, including, e.g., a vector, nucleic acid segment, cytokine, chemokine, toxin, or chemical compound. The vector can be any viral or non-viral vector as described in more detail below in the section regarding vectors for use in methods of the invention. Such animal models include the clinical manifestations or characteristics of atherosclerosis typically observed in human atherosclerosis or human atherosclerotic lesions.

The animal models of the present invention offer several advantages over known animal models of atherosclerosis. Existing animal models are essentially limited to uninjured or balloon-injured arteries of normolipidemic rats and pigs and balloon-injured arteries of hypercholesterolemic rabbits. See, e.g., Nabel et al., Annu. Rev. Physiol. 56:741-761 (1994); Guzman et al., Proc. Natl. Acad. Sci. USA 91:10732-10736 (1994); Rade et al., Nat. Med. 2:293-298 (1996); Simari et al., J. Clin. Invest. 98:225-235 (1996). Such known models have important limitations. Arterial lesions that develop in the absence of hyperlipidemia generally contain neither excess lipid nor macrophages, both central components of human atherosclerosis. Balloon-injured arteries lack endothelium, a critical cell type in atheroma development. Moreover, balloon injury in hyperlipidemic rabbits causes severe arterial inflammation with endothelial loss, medial destruction, and exuberant macrophage infiltration. Weidinger et al., Circulation 84:755-767 (1991); Faxon et al., Arteriosclerosis 2:125-133 (1982). Such features are uncharacteristic of lesions found normally in human atherosclerosis at any stage. See, e.g., Stary et al., Circulation 89:2462-2478 (1994); Stary et al., Circulation 92:1355-1374 (1995). Consequently, such models are not useful for studying human atherosclerosis.

The animal models of atherosclerosis in the present invention are particularly advantageous over such known models of atherosclerosis because they comprise an atherosclerotic lesion that is representative of human atherosclerotic lesions. The animal models described herein have several features in common with human atherosclerosis. For example, as in typical human atherosclerotic lesions, the lesions of the instant animal models develop below intact endothelium. Second, early lesions are characterized by macrophages that adhere to and migrate within an otherwise histologically normal artery wall. Third, phenotypically modulated smooth muscle cells and excess intimal lipid deposits are prominent components of the lesions in these models. Fourth, lesion growth is cholesterol-dependent in the novel models described herein. Fifth, inflammation (assessed by T cell infiltration and upregulation of vascular cell adhesion molecules) is a prominent feature in these models. Additional features, as reflected in the data presented herein, demonstrate the utility of the model; for example, the lesions are reliably localized at the site of infusion, facilitating sampling protocols, and intragroup variability is low, thus permitting use of only 5-8 arteries per group to detect statistically significant 2-3-fold differences.

The animal models of the present invention are also advantageous over known animal models of atherosclerosis because they permit the study of the effect of genes that prevent or retard the development of atherosclerosis at the level of atherosclerotic vessel wall (e.g., artery wall). In existing animal models, genes that prevent or retard the development of atherosclerosis cannot be studied at the level of artery wall. With existing gene transfer models, only those genes that reverse atherosclerosis can be studied, since such models can be used only after atherosclerotic lesions have been developed. With the animal models of the present invention, the initiation and development of early lesions can be studied and modulated. Furthermore, animal models of the present invention permit the development and testing of prophylactic measures and treatments, because test agents (including transgenes) can be delivered to the models prior to potential atherosclerotic lesion development or during such development. For example, modifier genes can be inserted as expression cassettes and the effect of local overexpression of their protein products can be revealed beginning precisely at the time of lesion initiation. Systemic overexpression, embryonic lethality, and developmental compensation—potentially confounding phenomena in germ line transgenic systems—are either highly unlikely or impossible in animal models of the present invention.

Animal models of the present invention are particularly useful for detecting specific therapeutic or prophylactic agents (test agents) for treating and/or preventing diseases and conditions linked to vascular disease, in particular atherosclerosis and cardiovascular diseases, such as myocardial infarction, sudden death, angina, heart attack, stroke, and other forms of heart and vascular disease.

The animal models of the invention include any non-human mammal, including rabbit, mouse, pig, or non-human primate (e.g., monkey). The rabbit model was selected as a preferred animal model for the reasons described above. See, e.g., the Examples section below, which describes production and use of the rabbit model of atherosclerosis in more detail. Rabbit models are especially useful for performing studies using vectors to screen for therapeutic and prophylactic test agents for an activity on atherosclerosis and (adenovirus-mediated) gene transfer therapy to treat atherosclerosis. See, e.g., the Examples below, which describe production and use of the rabbit model of atherosclerosis in more detail.

IV. Methods of Producing Non-Human Mammalian Models of Atherosclerosis

The present invention provides methods of producing a non-human mammalian model of atherosclerosis. Such methods are useful in producing appropriate models of the human atherosclerosis which can be used, in particular in screening methods and gene therapy methods to investigate the effects of test agents on atherosclerosis. Such methods comprise: (a) administering an atherogenic diet to the non-human mammal; (b) isolating a blood vessel or other vascular element of the non-human mammal; (c) delivering a proinflammatory agent to the blood vessel or other vascular element of the non-human mammal; and (d) maintaining the non-human mammal for a time sufficient for an atherosclerotic lesion to develop in the blood vessel or other vascular element, thereby producing a model of atherosclerosis. Such methods of producing atherosclerotic animal models include those described in more detail in the Examples section below.

In some such methods, the proinflammatory agent comprises a vector, cytokine, or chemokine which acts directly or indirectly on the blood vessel of the subject to which it is delivered or administered. The vector may be any viral or non-viral vector as described in more detail below in the section regarding vectors for use in methods of the invention. In addition, in some such methods, the viral vector comprises an adenoviral vector, retroviral vector, adenoassociated viral vector, alphaviral vector, or herpes simplex viral vector.

Cytokines are hormone-like low molecular weight proteins, secreted by many different cell types, which regulate the intensity and duration of immune response and are involved in cell-to-cell communication. See STEDMAN'S MEDICAL DICTIONARY, supra. Chemokines are chemotactic cytokines—that is, cytokines that promote chemotaxis. Chemotaxis is the movement of cells or organisms in response to chemicals, whereby the cells are attracted or repelled by substances exhibiting chemical properties. See STEDMAN'S MEDICAL DICTIONARY, supra.

The vascular element which is isolated for observation and analysis in such methods of production of the animal model may comprise a blood vessel, such as an artery or vein, or other vascular element. Methods of the present invention can be practiced using a variety of blood vessels, including veins, such as saphenous veins or other veins used as bypass conduits, or arteries, such as the carotid artery, femoral artery, brachial artery, radial artery, gastroepiploic artery, iliac artery, innominate artery, aorta, coronary artery, or vertebral artery, or other vessels, as set forth in STEDMAN'S MEDICAL DICTIONARY, supra, at 136-149. Known, standard procedures for surgically isolating all such blood vessels can be employed for use in the methods of the present invention. See, e.g., Newman et al., J. Clin. Invest. 96:2955-2965 (1995) for a procedure for isolation of femoral arteries.

Any non-human mammal may be used in such methods. In one aspect of the invention, the non-human mammal comprises a rabbit, mouse, pig, or non-human primate (e.g., monkey). The rabbit is among the preferred non-human mammals for use in methods of producing such animal models. For details about methods of producing rabbit models of atherosclerosis, see the Examples below.

The present invention also provides methods of inducing development of experimental atherosclerotic lesions in a non-human mammal, including those outlined above. Such methods comprising: (a) administering to the non-human mammal an atherogenic diet; (b) isolating a blood vessel of the non-human mammal; and (c) introducing a proinflammatory agent into the blood vessel, wherein administration of the atherogenic diet and introduction of proinflammatory agent in the artery induces development of an atherosclerotic lesion in the blood vessel. Such methods can be performed as described in detail in the Examples section below.

V. Screening Methods of the Invention

The present invention provides in vivo and ex vivo methods of screening a test agent for an activity on atherosclerotic lesion development in a non-human mammal. Such methods are useful for screening an agent for an activity on the development of atherosclerotic lesions in vivo or ex vivo and for identifying which such agents would be useful in therapeutic or prophylactic methods of treating atherosclerosis and related vascular disorders. Such methods comprise: administering to the non-human mammal an atherogenic or cholesterol-rich diet, surgically isolating an artery, vein, or other vascular element of the non-human mammal, delivering a proinflammatory agent to the artery, vein, or other vascular element of the non-human mammal, delivering the test agent to the non-human mammal, and monitoring a property of the artery, vein, or other vascular element to indicate an activity on atherosclerotic lesion development in the artery, vein, or other vascular element. In some such screening methods, the proinflammatory agent and test agent are delivered to the non-human mammal simultaneously. In other such methods, the test agent is delivered to the non-human mammal before the proinflammatory agent is delivered to the non-human mammal. Detailed examples illustrating some such screening methods (including a method to screen for the effects of transduced FasL) are presented in the Examples section below.

In one aspect, in monitoring the artery, vein, or other vascular element for an activity or effect of the test agent on the development of atherosclerotic lesion(s), a response in the artery, vein, or other vascular element which indicates an activity or effect or atherosclerotic lesion development is detected.

A variety of activities relating to atherosclerotic lesion development can be monitored, detected, and analyzed. In another aspect of the invention, the activity is an increase or decrease in atherosclerotic lesion development. An increase or decrease in atherosclerotic lesion development can be identified and detected by observing, monitoring over time, and analyzing clinical manifestations and characteristics of atherosclerosis and atherosclerotic lesions, including those described in detail in the section entitled “Atherosclerosis: Development and Manifestations.” Atherosclerotic lesion development can be observed, monitored, and/or analyzed by, e.g., methods presented in the Examples section below.

Any non-human mammal may be used in such screening methods. In one aspect of the invention, the non-human mammal comprises a rabbit, mouse, pig, or non-human primate (e.g., monkey). The rabbit is among the preferred non-human mammals for use in such screening methods. See, e.g., Examples below.

The vascular element which is isolated for observation and analysis in such methods of production of the animal model may comprise an artery, vein, or other vascular element. The vein may comprise saphenous veins or other vein used as a bypass conduit. The artery may comprise a carotid artery, femoral artery, brachial artery, iliac artery, radial artery, gastroepiploic artery, innominate artery, aorta, coronary artery, or vertebral artery, or other artery as set forth in STEDMAN'S MEDICAL DICTIONARY, supra, at 136-149.

A variety of proinflammatory agents may be used to induce, stimulate, and/or produce an inflammatory response or effect in a vascular element (e.g., artery or vein) of the non-human mammal. As noted above, proinflammatory agents include, among other things, vectors, nucleic acid segments, cytokines, chemokines, toxins, or chemical compounds. Such vectors can be viral or non-viral vectors, as described in more detail below in the section regarding vectors for use in methods of the invention. For example, in some such methods, the viral vector comprises an adenoviral vector, retroviral vector, adenoassociated viral vector, alphaviral vector, or herpes simplex viral vector.

A wide range of test agents can be screened and tested for an activity on atherosclerotic lesion development and atherosclerosis using the screening methods of the present invention and the animal model of atherosclerosis described herein. The animal model of the present invention provides a vehicle for selection of effective agents from among a large battery of known and novel compounds. For example, an array of potential therapeutic and/or prophylactic agents can be screened and tested. Potential test agents include nucleic acid segments, genes that may express a therapeutically or prophylactically useful peptide or protein for atherosclerosis or its lesion development, genetic agents, gene therapy agents, viruses, pharmaceutical compounds, pharmacological compounds, drugs, toxins, chemical substances and chemical compounds, and natural products. Such agents may be screened and tested for a positive or negative effect of atherosclerotic lesion development. Such effects are determined by monitoring the development, clinical manifestations, characteristics, and properties of such lesions, as described more specifically above and in the Examples section below.

In some screening methods of the present invention, the test agent comprises a gene which is expressed in the blood vessel upon delivery of the vector to the blood vessel. In some such methods, the gene encodes a therapeutically or prophylactically useful protein or peptide for treating atherosclerosis or atherosclerotic lesions.

In some screening methods of the invention in which the proinflammatory agent is a vector, the vector comprises a gene which encodes the test agent, wherein said gene expresses the test agent in the blood vessel upon delivery of the vector to the blood vessel. In some such methods, the test agent encoded by the gene comprises a therapeutically or prophylactically useful protein or peptide for treating atherosclerosis or atherosclerotic lesions. For example, retrovirus at a concentration of from about 10⁴ to about 10⁹ colony forming units per milliliter (cfu/ml) can be used in methods of the present invention; adenovirus at a concentration of from about 10⁴ to about 10¹² plaque forming units per milliliter (pfu/ml) can be employed in the methods of the invention.

The dosage and delivery of any particular test compound or agent to be screened or any potential therapeutic or prophylactic compound or agent to be used for therapeutic or prophylactic treatment of atherosclerosis can be determined on the basis of well-established guidelines for preparing pharmaceutically active compounds. Test compounds and agents can be administered, for example, intravenously, intradermally, intramuscularly, topically, orally, or by any other pharmaceutically effective route of administration. Using the animal models produced according to methods of the invention, an investigator can evaluate the activity or effect of a test agent against the development of atherosclerotic lesions (including prevention and retarding of such lesions)—even in subjects who do not yet manifest the disease or such lesions, but are at risk for developing them. Such agents may include, among other things, chemical-type pharmaceuticals, genetic agents for gene therapies, and vaccinations. Methods of evaluating the results of laboratory tests of proposed test agents, including therapeutics and prophylactics, are known.

The method and route of delivery of test agents and proinflammatory agents, and other agents of the invention is determined by the disease or clinical indication and the site where treatment is required. For topical application, it may be desirable to apply the test agent or proinflammatory agent, or other agent at the desired local site (for example, by applying topically to a blood vessel manifesting atherosclerotic disease or at risk for such disease). For some applications, it may be desirable to administer the test agent or proinflammatory agent by infusion into a desired delivery site (e.g., artery or vein) or systemically.

For other applications and indications, the test agent or proinflammatory agent may be delivered by intravenous, intraperitoneal, intramuscular, subcutaneous, intranasal, intradermal injection, intrabronchial instillation (e.g., by using a nebulizer), transmucosal, systemic, transdermal (e.g., with a lipid-soluble carrier in a skin patch), oral, and/or gastrointestinal delivery (e.g., with a capsule or tablet). During or after cardiovascular surgery (e.g., cardiac bypass surgery, angioplasty, and the like) or peripheral vascular surgery, test agents and/or proinflammatory agents, or other such agents may be administered in an intravenous bolus injection (or by perfusion).

One or more test agents or proinflammatory agents can be administered together or with other vectors or agents in combination screening methods and combination gene therapy methods. For example, one or more subject vectors may be administered in combination with a pharmacological agent (such as, e.g., ganciclovir or 5-fluorocytosine) to enhance the efficacy of the expressed gene (e.g., thymidine kinase or cytosine deaminase). These vectors and agents are administered to a hypercholesterolemic non-human mammal or subject to induce or produce atherosclerotic lesions and to permit screening of the effect of the pharmacological agent on development of such lesions.

With screening methods of the invention, a number of arterial or venous properties which indicate an effect or activity of a test agent on atherosclerotic lesion development (e.g., atherosclerotic “responses”) in the subject animal can be monitored. Such arterial or venous properties include a clinical manifestation, characteristic, symptom, or event that occurs in or is observed in or associated with the blood vessel being monitored for atherosclerotic lesion development. Such arterial or venous properties and/or atherosclerotic responses include, among other things, the proliferation of smooth muscle cells, accumulation of inflammatory T cells, and macrophages in the intima of the artery or vein, an increase in inflammation in the artery, increase in lesion size, increase in matrix accumulation or lipid accumulation, and intimal hyperplasia.

In addition, increases in vascular cell adhesion molecule-1 (VCAM-1) and intercellular adhesion molecule-1 (ICAM-1) in the artery or vein is another property which can be monitored, since vascular expression of VCAM and ICAM is associated with arterial pathology in animals and humans. Newman et al., J. Clin. Invest. 96:2955-2965 (1995); Bevilacqua, Annu. Rev. Immunol. 11:767-804 (1993); Printseva et al., Am. J. Path. 140:889-896 (1992); Poston et al., Am. J. Path. 140:665-673 (1992); O'Brien et al., J. Clin. Invest. 92:945-951 (1993). VCAM-1 and ICAM-1—when expressed by vascular endothelium—are believed to participate in the adhesion and consequent entry of mononuclear leukocytes into the arterial wall. Id. Increases in VCAM-1 expression is one of the earliest detected vascular changes, occurring prior to monocyte infiltration. Id.; Li et al., Arterioscler. Thromb. 13:197-204 (1993). In addition, increased SMC expression of ICAM-1 and VCAM-1 is also associated with established and developing vascular disease.

Methods and techniques for identifying, monitoring, and analyzing increases in smooth muscle cells, macrophages, T cells in blood vessels (e.g., arteries and/or veins), changes in endothelial cells, and increased expression of VCAM-1 and ICAM-1 are known to those of skill in the art. Such methods include, for example, those methods described in the Examples section below and described in Braunwald, supra, including Chap. 35 (3d ed. 1988), and V. Fuster et al., ATHEROSCLEROSIS AND CORONARY ARTERY DISEASE (Lippincott-Raven Pub., 1996), each of which is incorporated herein by reference in its entirety for all purposes.

In yet another aspect, the invention provides methods of screening a gene for a therapeutic or prophylactic activity on atherosclerotic lesion development in a non-human mammal, which comprises: (a) administering an atherogenic diet to the non-human mammal; (b) isolating a blood vessel of the non-human mammal; (c) delivering a vector to the blood vessel, wherein the vector comprises a gene and expresses the gene in the blood vessel; and (d) monitoring a property of the blood vessel which indicates a therapeutic or prophylactic activity of the gene on atherosclerotic lesion development in the blood vessel, including, e.g., anti-atherosclerotic activity and a decrease in atherosclerotic lesion development as described above and shown in more detail in the Examples section below. Such methods are useful in identifying genes that may be effective in prophylactic and/or therapeutic methods and gene therapies for treating atherosclerosis in animals, including humans and non-human mammals. As noted above, a variety of arterial properties can be monitored to ascertain the effect and activity of the gene on such lesion development.

In another aspect, the invention provides methods of screening a test agent for a capacity to induce or inhibit atherosclerotic lesion development in a subject exposed to the agent. Such methods comprise exposing the subject to or contacting the subject with the test agent and detecting a response in the subject, or monitoring a property of a blood vessel of the subject (e.g., a blood vessel to which the test agent has been delivered or which has been contacted with the agent), to indicate, detect, or ascertain an effect of the agent on atherosclerotic lesion development. As with other screening methods of the invention, including those set forth in the Examples below, such results can be compared to a control application in which the subject is not exposed to or contacted with the test agent. Such comparison facilitates determination of the extent of atherosclerotic lesion development and whether the agent induces or inhibits atherosclerotic lesion development in the subject.

VI. Gene Therapy Methods of the Invention

The present invention also provides in vivo and ex vivo gene therapy methods in which a gene therapy agent, vector, or nucleic acid segment comprising a gene is introduced directly into target cells of blood vessel walls (e.g., walls of artery or veins) of isolated blood vessels of animal models of atherosclerosis produced by methods of the present invention. In ex vivo methods, the target cells or tissues are removed from the subject, directly manipulated, and then re-implanted in the subject. In vivo and ex vivo gene therapy methods of the present invention are useful in elucidating the role of individual genes in local arterial biology and for investigating potential of recombinant genes and proteins for use in therapeutic methods for the treatment of arterial diseases. Specific arterial tissue sections in which genes are expressed can be identified. Such in vivo and ex vivo gene therapy methods permit effective and efficient transfer and genetic expression (as measured by the amount of gene product present in the selected arterial tissue sections) and permit site-specific and localized expression of genes in vivo and ex vivo. With these methods, local pathogenic processes involved in atherosclerosis can be successfully investigated and approaches for treating and preventing atherosclerosis in humans can be developed. Such gene therapy methods are useful for therapeutic and prophylactic management of human atherosclerotic disease. Gene therapies of the present invention are also useful in treating and/or preventing diseases associated with vascular disorders and atherosclerosis, such as heart attacks, angina, stroke, heart attacks, peripheral vascular disease, and other forms of heart and vascular diseases.

In one aspect, the present invention provides methods of gene therapy for prophylactic or therapeutic treatment of atherosclerosis in a subject in need of such treatment. Such methods are useful in treating atherosclerosis and related conditions in subjects at risk of or suffering from atherosclerosis or a related vascular disease. Such methods comprise administering to, delivering to, or introducing into the subject, such as a non-human mammal or a human, a gene therapy agent or vector which, for example, comprises a gene which encodes a therapeutically or prophylactically useful peptide or protein for atherosclerosis. Alternatively, the nucleic acid segment may comprise a gene which encodes a therapeutically or prophylactically useful peptide or protein for atherosclerosis. For example, the gene or nucleic acid segment may encode a product that prevents or retards the development of atherosclerotic lesions or related symptoms of atherosclerosis. The gene is expressed in the subject upon introduction or delivery to the subject and the gene product is synthesized, thereby promoting prophylactic or therapeutic treatment of atherosclerosis in the subject. Such vectors may be delivered to or administered to the subject by a variety of methods, including, among other methods, those described below and above. For example, the vector may be effectively delivered to the subject by infusion into a surgically isolated blood vessel of the subject, as described here and in more detail below in the Examples below.

Gene therapy agents (e.g., vectors and nucleic acid segments comprising desired genes) can be introduced or delivered to the subject to treat atherosclerosis and related lesions in the vascular system without systemic dilution or undesired effects on other organs. Agents can comprise naked DNA or DNA compositions for delivery of genetic information in vivo (or ex vivo) or cells which have been genetically modified in vitro or (ex vivo). Genetic information can be transferred by using a number of known methods. For example, DNA may be delivered by physical means, such as, among other things, infusion, microinjection, electroporation, biobalistic or particle bombardment, jet injection, and other introduction methods. In another aspect, DNA can be delivered by chemical means, using calcium phosphate, DEAE dextran, polylysine conjugates, liposomes, virion-like particles, intra-cellular-targeting ligands, receptor-mediated uptake systems such as transferrin, and others. In another aspect, DNA can be delivered by biological means, including retroviral vectors, such as those described below and above, including, among others, adenoviral vectors and adenoassociated viral vectors, herpes simplex vectors, and alphaviral vectors. For a general discussion of gene therapy, gene therapy methods, and vectors for use in gene therapy, see PCT International Publication No. WO 98/13485, which is incorporated herein by reference in its entirety for all purposes.

The amount of gene therapy agent (e.g., vector) to be delivered or introduced into a subject depends on the particular application. When a vector is used to deliver a gene therapy agent, such as, for example, a gene which encodes a therapeutically or prophylactically useful protein or peptide for atherosclerosis, such vector should be delivered at a concentration of between from about 10⁴ to about 10⁹ cfu/ml for retroviruses and from about 10⁴ to about 10¹² pfu/ml for adenoviruses can be used in the methods of the invention. A total amount of injected, delivered or infused fluid may range from about 0.1 ml to about 15 ml. The agent should achieve effective transfer in from about 1 minute to about 30 minutes. A detailed discussion of gene therapy vectors is provided in the section regarding vectors for use in methods of the invention below.

In some embodiments of the invention, the gene therapy agent (e.g., vector or nucleic acid segment) is mixed with a known pharmaceutically acceptable carrier such as viscous biocompatible polyol or other such suitable carrier, including those discussed in Human Gene Therapy 6:41-53 (1995). Such carriers may facilitate high rates of transduction.

In another aspect, the invention provides gene therapy methods which are useful for compensating for a defect or mutation in an endogenous gene by integrating a functional copy of the (same) gene into the host chromosome of a subject in need of such treatment. The inserted gene may replicate with the host DNA and is expressed at a level to compensate for the defective gene. Atherosclerosis is amenable to treatment by this approach. An exogenous functional gene which is intended to compensate for the genetic defects can be introduced into the subject by a variety of methods. In one method, cells are removed from a subject suffering from atherosclerotic disease and contacted with a vector containing a gene which expresses a therapeutically or prophylactically useful peptide or protein for atherosclerosis and its lesions. Cells can be removed from the vascular tissue (e.g., blood vessel) in which disease symptoms are observed. Cells are contacted with the vector containing the gene of interest. After integration of the vector into the cellular genome, and optionally, selection, cells are reintroduced into the subject. If desired, the gene provided by the vector can be delivered to the same site (e.g., blood vessel) as is occupied by the defective gene that it is intended to compensate.

In another aspect, the invention provides gene therapy methods which are useful for augmenting expression of an endogenous gene that is present in a subject, but that is expressed at a level that is inadequate to prevent or reverse disease. An exogenous functional copy of the same gene present in the subject is introduced into the subject by a variety of methods, including those outlined above, and may integrate into the host chromosome of the subject in need of such treatment. The inserted gene may replicate with the host DNA and is expressed at a level sufficient to compensate for the low expression of the endogenous gene. Atherosclerosis and atherosclerotic lesions can be treated by this approach.

In yet another aspect, the invention provides gene therapy methods which are useful in compensating for a defect or mutation in an endogenous gene in a subject (e.g., the endogenous gene is missing in the subject or is expressed at an abnormally low level) by integrating a different gene from another species or an engineered gene into the host chromosome of the subject in need of the treatment. Such methods are useful in counteracting the defects of the endogenous gene. The introduced gene augments or compensates for the defective endogenous gene by its expression of a useful product (e.g., peptide); such a gene can be introduced into the subject by a variety of methods, including those discussed above. Atherosclerosis and atherosclerotic lesions can be treated by such methods.

In another aspect, the invention provides gene therapy methods which are useful for directing and localizing expression of a gene at a particular location or tissue site in the body of the subject (i.e., ectopic expression). That is, by using the methods of the invention, the desired gene is introduced into the subject (by using methods described above) and expressed at a location or site where the gene is not normally expressed or where augmented expression is desired. Atherosclerosis and atherosclerotic lesions are amenable to treatment with such methods.

A gene or nucleic acid segment or sequence comprising a gene which is useful for employment in such gene therapy methods can be identified by the screening methods of the present invention. Such screening methods can also be used to identify gene products (e.g., peptide and proteins) encoded by genes that act as therapeutically or prophylactically useful compounds for preventing or treating atherosclerosis. Methods of isolating, purifying, and making genes and/or nucleic acid segments or sequences comprising desired gene sequences are well known to those of skill in the art. See, e.g., Sambrook et al., Molecular Cloning, A Laboratory Manual, (Cold Spring Harbor Laboratory, Cold Spring Harbor, N.Y., 2nd ed. 1989).

A. Effects of FasL Expression on Atherosclerosis

The mammalian models, screening methods, and gene therapy methods of the present invention are useful for studying genes that may modify the atherosclerotic process. As an example, we investigated the role of Fas ligand (FasL) in atherosclerotic lesion development. Apoptosis is mediated, at least in part, by a cell surface receptor protein known as the Fas antigen receptor (“Fas”). Fas is a transmembrane protein belonging to the tumor necrosis factor (“TNF”)/nerve growth factor (“NGF”) receptor family of proteins which is expressed in a variety of tissues and human cells. Tanaka et al., Nature Med. 2:317-322 (1996); Tanaka et al., EMBO J. 14:1129-1135 (1995); Kayagaki et al., J. Exp. Med. 182:1777-1783 (1995). Fas has been found to transduce extracellular signals into a cell and thus it can mediate or trigger apoptosis. See, e.g., Itoh et al., Cell 66:233-243 (1991). Because Fas is expressed on the cell surface, its action is believed to be regulated by interacting with or binding to FasL, which is a cell surface protein. FasL is expressed by a variety of cells, including endothelial cells, and is present in human atherosclerotic lesions. Tanaka et al., Nature Med. 2:317-322 (1996); Kayagaki et al., J. Exp. Med. 182:1777-1783 (1995). FasL has been found to mediate and induce apoptosis by binding to Fas. Takahashi et al., Int'l Immunol. 6:1567-1574 (1994); Abbas, Cell 84:655-657 (1996).

By inducing apoptosis in infiltrating leukocytes, FasL could be atheroprotective. Alternatively, by activating proinflammatory pathways, FasL may promote the formation of atherosclerotic lesions. To resolve this question, we explored the expression of FasL using our model of primary atherosclerotic lesion development in uninjured carotid arteries of hypercholesterolemic rabbits (details of which are described above). Isolated carotid arteries of hypercholesterolemic rabbits were infused with either a replication-defective adenoviral vector encoding FasL (“AdFasL”) or a control vector lacking the FasL transgene (AdNull). Methods for inducing and identifying lesions, analyzing intimal:medial ratios, and transducing cells were performed according to the methods of the invention, as described herein and in detail elsewhere in this application.

Intimal:medial ratios of the rabbit carotid arteries were analyzed 7, 14, and 28 days, respectively, after infusion into the arteries of either control vector AdNull or an adenoviral vector containing Fas ligand (AdFasL). Intimal lesion formation was significantly accelerated by infusion of AdFasL. Intima:Media ratios in AdFasL-transduced arteries were greater than in AdNull-transduced arteries at 7 days (0.22±0.03 for AdFasL infusion vs. 0.07±0.02 for AdNull infusion; P<0.01) and 14 days (0.22±0.02 vs. 0.07±0.02; P<0.01), but not 28 days (0.30±0.03 vs. 0.28±02; (P=NS) after vector infusion. Endothelial cell overexpression of FasL did not affect macrophage accumulation, but inhibited T cell infiltration and eliminated endothelial cell VCAM-1 expression. Infusion of AdFasL was associated with increased numbers of proliferating, α-actin-negative smooth muscle cells in the intima (approximately 30% to 40% of intimal cells). FasL-mediated acceleration of atherosclerosis was associated with a moderate antiinflammatory effect (on T cells and VCAM-1 expression) but an apparent mitogenic (rather than pro-apoptotic) effect on SMC leading to enhanced lesion growth. Using western blotting, we found no evidence of FasL expression in normal, nondiseased carotid arteries.

These data show that local overexpression of FasL in endothelium accelerates intimal lesion formation in this animal model. Notably, this acceleration occurred despite essentially complete elimination of T cells from the intimal lesions. Fas ligand expression had no effect on macrophage density, and there was a significant increase in the rate of smooth muscle cell accumulation in the media. This accumulation was associated with an increase in intimal smooth muscle cell proliferation. There was no evidence of increased apoptosis in the blood vessel wall, as determined by terminal deoxynucleotidyl transferase-mediated dUTP nick end labeling (TUNEL) staining of arteries harvested at varying times after gene transfer. The results suggest that FasL-mediated suppression of T cell infiltration is not atheroprotective and that activation of FasL signalling pathway may contribute to atherosclerotic lesion development. These results suggest a novel, potentially atherogenic role for Fas ligand expression in the vessel wall. The role of FasL in the artery wall is pathogenic rather than protective.

VII. Vectors for Use in Methods of the Invention

A variety of vectors may be employed in all methods of the present invention—including methods of producing mammalian models of atherosclerosis, methods of screening agents for activities on atherosclerotic lesion development, and gene therapy methods. As noted above and shown in the Examples which follow, both viral and non-viral vectors can be used. Examples of viral vectors include the adenoviral vector, retroviral vector, adenoassociated viral vector, alphaviral vector, or herpes simplex viral vector. Examples of non-viral vectors include plasmids, RNA, with or without agents used to increase their entry into cells.

In our methods of producing an animal model of atherosclerosis, we found adenoviral vectors to be effective vectors for the inducement in animals (e.g., rabbits) of atherosclerotic disease conditions which closely resemble those of humans. Adenoviral vectors have proinflammatory effects including neointimal hyperplasia, inflammation, and vascular cell activation. In particular, we found that the local infusion of AdNull (adenovirus lacking a gene or transgene) into surgically isolated rabbit carotid arteries of diet-induced hypercholesterolemic rabbits produced an appropriate model of atherosclerosis. As shown in detail in the Examples below, infusion of AdNull into such carotid arteries caused neointimal formation and inflammation. Hypercholesterolemia exacerbated this process, resulting in larger, macrophage-rich lesions resembling fatty streaks—including human fatty streaks. Notably, the arteries in the lesions produced by this method had a histologically intact endothelium and an intima comprising numerous smooth muscle cells as well as macrophages and T cells—as are observed in human atherosclerotic lesions.

In general, a gene therapy vector is an exogenous polynucleotide which produces a medically or diagnostically useful phenotypic effect upon the mammalian cell(s) into which it is transferred. A vector may or may not have an origin of replication. For example, it is useful to include an origin of replication in a vector for propagation of the vector prior to administration to a subject. The origin of replication can be removed before administration if the vector is designed to integrate into host chromosomal DNA or bind to host mRNA or DNA.

Vectors used in gene therapy methods of the present invention can be viral or nonviral. Viral vectors are usually introduced into a subject or mammal as components of a virus. Nonviral vectors, typically dsDNA, can be transferred as naked DNA or associated with a transfer-enhancing vehicle, such as a receptor-recognition protein, lipoamine, or cationic lipid.

Viral vectors include, for example, adenoviruses, retroviruses, alphaviruses, adenoassociated viruses, and herpes simplex viruses. Viral vectors typically comprise two components: a modified viral genome and a coat and/or envelope structure surrounding it. See generally Smith, Annu. Rev. Microbiol. 49:807-838 (1995). Some viral vectors are introduced in naked form or coated with proteins other than viral proteins. Most vectors have coat structures similar to a wildtype virus, which enables the viral nucleic acid to bind and enter the subject's target cells. The viral nucleic acid in a vector employed for gene therapy differs in several ways such that it is changed to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Vectors of the present invention may contain a DNA fragment of interest that is to be screened for an activity or effect on atherosclerotic lesion development. Such vectors are constructed by inserting the DNA fragment of interest into the vector, according to standard methods. The DNA fragment of interest can be cDNA, genomic, minigene (genomic with one or more introns omitted), synthetic or a hybrid of any of these. Genomic sequences can lead to higher levels of expression. The fragment often encodes a protein. The nature of the protein depends on the intended use. In gene therapy applications for the therapeutic or prophylactic treatment of atherosclerosis, the protein is a functional expression product that can compensate for the defective expression product of a mutant gene (e.g., a gene that renders a subject more susceptible to atherosclerotic lesion development or other manifestations of atherosclerosis) or augment the expression of an endogenous gene (as described above). Additionally, in some gene therapy applications of the present invention for the therapeutic or prophylactic treatment of atherosclerosis, the protein is a functional expression product that compensates for the defective expression product of a mutant gene (e.g., a gene that makes the subject more susceptible to atherosclerotic lesion development or other manifestations of atherosclerosis). In other such gene therapy applications for treating atherosclerosis, the protein is a functional expression product that augments the expression of an endogenous gene in the subject which protects against or is involved in the development of atherosclerosis, but which is expressed at a low level. In other gene therapy applications for treating atherosclerosis, the protein is a functional expression product that is expressed ectopically at a location or tissue site in the subject at which is not normally expressed.

A DNA fragment can encode the coding sequence of a wildtype form of the gene. Alternatively, the expression product can be an antisense sequence exhibiting complementarity to the genome of a microorganism. In some applications, more than one fragment of interest is inserted, and the vector is thus capable of expressing several peptides or proteins. Such peptides or proteins may be selected to have a therapeutic or prophylactic activity on atherosclerotic development. Such activity includes a decrease or increase in atherosclerotic lesion development. Such activity is detected by monitoring a property of the blood vessel (e.g., increase in SMC proliferation, increase in inflammatory T cells or macrophages, and other manifestations of atherosclerosis as described herein).

If the DNA fragment of interest is a protein-coding sequence, the sequence is operably linked to a promoter and preferably an enhancer. The promoter and enhancer should be functional in the cell or tissue type in which it is desired that expression be obtained. Some promoter and enhancers are relatively nontissue specific (e.g., regulatory sequences derived from immunoglobulin genes, SV40, adenovirus, bovine papilloma virus, cytomegalovirus). Others regulatory sequences promote expression more effectively in a specific tissue type. For example, casein regulatory sequences promote expression in mammary tissue, albumin regulatory sequences in liver, ax-actin sequences in muscle and protamine sequences in spermatids.

Vectors for use in methods of the present invention can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. Additionally, vectors used in methods of the present invention are typically delivered by topical application, either under direct vision or via a catheter-based approach. Such vectors can also be infused locally or delivered topically to the blood vessel.

Alternatively, vectors for use in methods of the present invention can be delivered systemically or to a particular target tissue or site in the body of the subject. Such vectors typically include or incorporate a molecular “address” which assists in directing the vector to the desired target tissue or site (e.g., a vector may include a binding element which binds to a particular binding site on a specific target site or tissue in the subject).

The present invention is further illustrated by the following examples. These examples are merely to illustrate aspects of the present invention and are not intended as limitations of this invention.

### EXAMPLES

### Example 1

### Rabbit Model of Atherosclerosis

Adult male New Zealand White rabbits (Charles River Laboratories, Montreal, Quebec, Canada) weighing between about 2.5 to 3.5 kilogram (kg) were employed as animal models. The animals were housed individually in stainless steel, wire-bottomed cages in a room with a 12-hour light-dark cycle.

Hypercholesterolemia was in induced in some rabbits by feeding the rabbits 100 grams/day (gm/day) of rabbit chow containing 0.25% cholesterol and 3% soybean oil (Ziegler Bros.). Plasma cholesterol and triglyceride levels in the blood plasma of a subject rabbit were determined on a weekly basis using a clinical chemistry analyzer system (Abbott Spectrum, Abbott Laboratories). After two weeks on this diet, dietary adjustments were made based on plasma cholesterol levels. Specifically, rabbits with a plasma cholesterol concentration of less than 400 milligram/deciliter (mg/dl) after two weeks of the feeding protocol were continued on a diet of 0.25% cholesterol and 3% soybean oil diet. Rabbits having plasma cholesterol concentration levels between 400 and 500 mg/dl after two weeks of the feeding protocol were given a different diet comprising 0.125% cholesterol and 1.5% soybean oil. Rabbits having plasma cholesterol levels greater than 500 mg/dl after two weeks were fed normal chow for 4 days and were then placed on a diet comprising 0.125% cholesterol and 1.5% soybean oil. After 4 weeks, approximately 75% of all rabbits fed according to this protocol displayed blood plasma cholesterol levels ranging from 400 to 700 mg/dl. Rabbits with cholesterol levels in this range (e.g., 400 to 700 mg/dl) were entered into the atherosclerotic lesion development study and underwent bilateral carotid surgery. Rabbits with cholesterol levels outside this range were withdrawn and were not used in the study. Using this protocol, cholesterol levels at the time of infusion of vector or virus buffer storage solution (without vector) were 563+/−25 (Mean+/−Standard error; n=13). In rabbits undergoing vessel harvest at 4 weeks (n=12), cholesterol levels were 683+/−96.

The amount of cholesterol administered to the rabbits in the present study was lower than the high-cholesterol diet that is typically given to rabbits (e.g., 1-2% cholesterol). This amount of cholesterol fed to rabbits was selected so as to product atherosclerotic lesions in rabbits that most closely resemble those of humans. The diet administered to the rabbits as outlined above facilitated the production of atherosclerotic lesions similar to those observed in human atherosclerotic lesions; such lesions included smooth muscle cells/connective tissue lesions typical of human atherosclerotic lesions.

All animal procedures described herein and throughout this text were approved by the University of California, San Francisco Committee on Animal Research and conformed to the guidelines published in the Guide for the Care and Use of Laboratory Animals (Institute of Laboratory Animal Resources, Commission on Life Sciences, National Research Council, Washington: National Academy Press, 1996).

### Example 2

### Adenoviral Vector Preparation

Two adenoviral vectors were used in the screening and gene therapy experiments using rabbit models of atherosclerosis. A first generation adenoviral deletion mutant vector comprising an E1, E3-deleted adenovirus without an inserted gene or transgene was used in some methods of producing non-human mammalian models of atherosclerosis and methods of inducing development of atherosclerotic lesion in non-human mammals. In addition, the adenoviral vector was used in methods of screening test agents (including, e.g., nucleic acid segments which contain or do not contain genes to be expressed, proinflammatory agents, or pharmaceutical or pharmacologic agents) for an effect or activity on atherosclerotic lesions. This adenovirus, which was made in our laboratory by using procedure provided in Lee et al., Circulation Research 73(5):797-807 (1993), is termed “AdNull”. The construction of AdNull, a replication-deficient adenovirus lacking a transgene, has been described in Kang et al., Nature. Med. 3:738-807 (1997)). The E1 gene deletion renders the virus replication incompetent. This vector served as control vector to assess the effects of infusion of the adenovirus alone (i.e., without an inserted transgene) on the arterial wall of surgically isolated rabbit carotid arteries; such effects included arterial cell activation, mononuclear leukocyte infiltration, and intimal proliferation. Notably, second, third, and higher generation vectors can be used with methods of the present invention.

A second adenoviral vector, termed “AdFasL,” was made in our laboratory by using the procedure in Lee et al., Circulation Research 73(5):797-807 (1993). This adenoviral vector was used in gene therapy experiments to demonstrate the ability of the rabbit model system to express nucleic acid segments comprising genes (transgenes) inserted into adenoviral vectors and to evaluate specifically the effect of an inserted Fas ligand transgene on the development of atherosclerotic lesions. AdFasL was essentially identical to AdNull, except that it contained a murine Fas ligand (FasL) complementary DNA (cDNA) driven by the cytomegalovirus (CMV) immediate-early enhancer/promoter (see Kang et al., Nature. Med. 3:738-807 (1997)).

Both replication-deficient adenovirus constructs were constructed as previously described in Graham, F. L., and L. Prevec, Meth. Mol. Biol. 7:109-128 (1991). Propagation, purification, titration, and storage of the viral vectors were performed as described in Lee et al., Circulation. Res. 73(5):797-807 (1993). Ratios of total viral particles (concentration of adenoviral vectors was determined by measuring the optical absorbance of the adenoviral vectors at 260 nanometers (OD₂₆₀) as described in Mittereder et al., J. Virol. 70:7498-7509 (1996), incorporated herein in its entirety by reference for all purposes) to plaque-forming units (pfu) were less than 200 for all viral stocks. The absence of a replication-competent virus was confirmed in all viral preparations by a polymerase chain reaction (PCR)-based assay capable of detecting one E1-containing virus genome per 1×10⁶ vector genomes. Only PCR-negative preparations were used in in vivo methods.

For animal experiments, frozen viral vector stocks were thawed and used within 30 minutes of thawing. Vector stock solutions were diluted in Dulbecco's Modified Eagle Medium (DMEM) (Biofluids, Inc., Rockville, Md.) containing 1 milligram/milliliter (mg/ml) rabbit serum albumin (Sigma Chemical Co., St. Louis, Mo.) to achieve a final concentration of approximately 7.5×10¹¹ particles/ml. Control arteries were infused with a nonvirus-containing solution consisting of virus storage buffer (10 mM Tris-HCl pH 7.4, 1 mM MgCl₂, and 10% glycerol) diluted by the same dilution factor as the virus stock with DMEM containing 1 mg/ml rabbit serum albumin (to match the concentration of viral storage buffer in the diluted adenovirus preparations). (This nonvirus-containing solution is also termed a “vehicle” solution.) Concentrations of viral vector solution delivered included, for example, from about 10⁴ to about 10⁹ cfu/ml retrovirus or 10⁴ to about 10¹² pfu/ml adenovirus.

Rabbit aortic endothelial cells (EC) were a generous gift of Dr. Mahamad Navab (University of California, Los Angeles). EC were cultured on gelatin-coated flasks in EC growth medium (DMEM, low glucose (Gibco/BRL)) with 15% fetal bovine serum. Rabbit vascular smooth muscle cells (SMC) were isolated, as described in Pickering et al., J. Am. Coll. Cardiol. 20:1430-1439 (1992), from the iliac blood vessel of a New Zealand White rabbit (described above) and were grown in SMC growth medium (Medium 199 (Gibco/BRL) with 20% fetal bovine serum).

### Example 3

### Animal Surgery and In Vivo Adenoviral Vector Infusion

The New Zealand rabbits (described above) were sedated by intramuscular injection of ketamine (50 milligram/kilogram (mg/kg))(Fort Dodge Laboratories, Inc., Fort Dodge, Iowa) and xylazine (3 mg/kg) (Miles, Inc., Shawnee, Mission, Kans.). After endotracheal intubation, anesthesia was maintained with inhaled halothane (2% halothane). For infection prophylaxis, animals received enrofloxacin (2.27%, 0.2 ml/kg, intramuscularly (IM) before the skin incision once. Both common carotid arteries of a subject rabbit were exposed through a midline cervical incision, and the single side branch from each artery was coagulated and divided using an electrosurgical unit (Macan, Chicago, Ill.). After intravenous administration of heparin (100 Units/kg), one common carotid artery was isolated between atraumatic vascular clamps. Typically, a 3-cm long segment of the common carotid artery was clamped proximally and distally. An arteriotomy was performed just distal to the proximal clamp, and a 24-gauge catheter (Jelco, Critikon, Tampa, Fla.) was introduced through the arteriotomy. The surgically isolated arterial vessel segment was washed free of blood within the vessel lumen by flushing through the vessel via the catheter 1 mg/ml rabbit serum albumin in DMEM or another appropriate control solution. The catheter through which a vector or vehicle solution was to be infused into the segment was secured in place with a single 5-0 silk tie (ligature) to prevent egress of the adenovirus-containing solution.

Approximately 150 μl of the adenoviral vector solution or the virus buffer storage solution was infused into the vessel segment through the arteriotomy to distend the vessel segment to its normal physiologic caliber. The adenoviral vector solution consisted of 5×10⁹ pfu/ml adenoviral vectors diluted in DMEM containing 1 mg/ml rabbit serum albumin. The vehicle solution consisted of a virus storage buffer (10 mM Tris-HCl pH 7.4, 1 mM MgCl₂, 10% glycerol) diluted in DMEM containing 1 mg/ml rabbit serum albumin. The adenoviral vector was either an “empty” vector (i.e., lacking an inserted gene) or a vector containing an inserted gene. Infusion of vectors containing genes permitted an assessment of the effect of the gene on subsequent development of atherosclerotic lesions in the carotid artery. The solutions were infused to distend the artery to its normal physiologic calibre and were then allowed to incubate in situ for 20 minutes. After 20 minutes of incubation, the vector (or vehicle solution) solution was removed by aspiration back into the catheter, and the artery was briefly backflushed by release of the distal clamp and aspiration of blood into the syringe. Arteries were not intentionally injured during the infusion procedures. The catheter and silk tie were removed, the arteriotomy was repaired with 7-0 prolene suture, and blood flow was restored through the carotid artery by releasing the proximal and distal clamps. An identical procedure was then performed on contralateral carotid arteries in the rabbits. After the second artery had undergone infusion, the cervical wounds were closed in the subject animal in two layers. Rabbits were allowed to recover from anesthesia and replaced in their respective cages. The preoperative diet was resumed for each animal.

Vessels for organ culture were obtained essentially as described for frozen sections, except that the excised arteries were divided into 5 equal-sized rings, rinsed in 0.9% saline, and placed immediately on ice in M199 medium (Life Technologies, Gaithersburg, Md.) containing 0.4% fetal bovine serum. The 5 rings were rinsed an additional 4 times with medium, and placed in a single well of a 24-well plate containing 1 ml M199 with 0.4% fetal calf serum (FCS). The plate was kept in a 5% CO₂ incubator at 37° C. for 48 hours. Conditioned medium was then collected and vessel rings were snap frozen in liquid nitrogen. Both were stored at −80° C.

### Example 4

### Harvesting of Vessels in Rabbit Models

Following infusion of adenovirus into hypercholesterolemic and non-hypercholesterolemic rabbits, blood vessels (e.g., arteries) were harvested after a time period sufficient for atherosclerotic lesions to develop. This example illustrates procedures for harvesting of surgically isolated rabbit vessels that had been infused with vector or virus storage buffer solutions. Rabbit carotid arteries were harvested 2, 7, 14, 28, or 56 days after infusion of vector solutions or viral storage buffers solutions. As noted above, if desired, such vectors can contain transgenes that be tested for their prophylactic, therapeutic or other (e.g., detrimental) effects on induced atherosclerotic lesions. Vessels were either perfusion-fixed in situ and embedded in paraffin, snap-frozen in optimal cutting temperature (OCT) medium, a compound used to embed tissue for frozen sectioning. Perfusion fixation was carried out as follows: General anesthesia was induced in the rabbit and the abdominal aorta was exposed through a midline laparotomy. The aorta was ligated at the bifurcation and a 14-gauge catheter introduced proximal to the ligature. After intravenous administration of heparin (100 Units/kg), the animal was killed by an intravenous overdose of pentobarbital. A venotomy was made in the inferior vena cava and 300 ml of normal saline and one liter of 10% neutral-buffered formalin were infused sequentially into the aorta at a distending pressure of 90 millimeters (mm) Hg. The common carotid arteries were removed, placed in 10% neutral-buffered formalin for an additional 2 hours, and stored in 70% ethanol. Excised carotid arteries were then divided into 10 equal sized rings—each measuring approximately 3 mm in length. All 10 rings from each vessel were embedded side-by-side, sequentially, in a single paraffin block. 5 μm-thick sections of the rings were cut from each block for histochemical or immunohistochemical analysis to evaluate the extent of atherosclerotic lesion formation.

Vessel segments for frozen section analysis of atherosclerotic lesion formation were obtained essentially as described above, except that the perfusion protocol was omitted. The blood vessel was simply isolated between two vascular clamps and excised. After injection of pentobarbital, the common carotid arteries were excised and each blood vessel was divided into 4 rings of approximately 8 mm in length. The rings were rinsed in 0.9% saline, placed side-by-side in OCT medium, and snap frozen by immersion in isopentane and liquid nitrogen. Frozen ring sections (6-μm thickness) were stored at −20° C. until used in immunohistochemical studies.

### Example 5

### Histochemical and Immunohistochemical Staining of Vessels

This example describes histochemical and imnmunohistochemical staining procedures for visualizing blood vessels (e.g., arteries) which had been surgically isolated and infused with vector solutions or virus buffer storage solutions. Immunohistochemical and histologic analyses were performed on arterial sections to identify endothelial cells, smooth muscle cells, macrophages, T lymphocytes, expression of intercellular adhesion molecule-1 (ICAM-1), vascular cell adhesion molecule-1 (VCAM-1), neointimal hyperplasia, matrix deposition, and proliferating cell nuclear antigen (PCNA).

Serial paraffin-embedded vessel sections were stained with hematoxylin and eosin, Movat's pentachrome, Leder stain, and with monoclonal antibodies (mAb) specific for macrophages (mAb RAM-11, which recognizes a cytoplasmic antigen protein expressed by rabbit alveolar macrophages; 1:40 dilution; Dako Corp., Carpinteria, Calif.), smooth muscle α-actin (mAb HHF-35, which detects smooth muscle α-actin; 1:50 dilution; Enzo Diagnostics, Farmingdale, N.Y.), endothelial cells (mAb anti-CD31, JC/70A, which recognizes endothelial cells; 1:30 dilution; Dako Corp., Carpinteria, Calif.), or PCNA (1:40 dilution; Santa Cruz Biotechnology), which recognizes a molecule that is expressed only in nuclei of dividing cells). Serial frozen sections were stained with monoclonal antibodies specific to T cells (mAb anti-CD5, KEN5; 1:25 dilution; Spring Valley Labs, Woodbine, Md.), VCAM-1 and ICAM-1 (mAb Rb1/9 and mAb Rb2/3, recognizing rabbit VCAM-1 and ICAM-1, respectively, and 1:200 and 1:50 dilution, respectively; generous gifts of Dr. Myron Cybulsky of Harvard University, Boston, Mass.). Antibodies were applied to the prepared histologic sections.

Bound antibody was detected with the avidin-biotin peroxidase complex system (Vectastain ABC Elite kit, Vector Laboratories, Inc., Burlingame, Calif.) using 3-amino-9-ethyl-carbazole (Vector Laboratories, Inc., Burlingame, Calif.) as a chromogen to visualize antibody binding. Immunostained slides containing sections were counterstained with hematoxylin for subsequent analysis. Biotinylated anti-rabbit immunoglobulin, 1:200 dilution (Vector Laboratories, Inc., Burlingame, Calif.) was applied for about 30 minutes, followed by a 30-minute incubation in a Vectastain ABC-alkaline phosphatase reagent. The substrate 3-amino-9-ethyl-carbazole produced a red reaction product and sections were counterstained in methyl green. The specificity of primary antibody binding was confirmed both by omission of the primary antibody and by substitution of isotype-matched antibodies, if available.

Following immunochemical staining, sections were evaluated with light microscopy and scored for primary antibody reactivity by two independent observers, each of whom was blinded to the identity of the sections (i.e., each observer did not know the experimental treatment given to the rabbit whose tissue they examined). Antibody activity on each section was scored with the aid of a semiquantitative scale of staining intensity of 0 to 4, as described in Newman et al, J. Clin. Invest. 96:2955-2965 (1995), in FIGS. 5A-5D above, and in Example 8 below.

### Example 6

### Terminal Deoxynucleotidyl Transferase-mediated dUTP Nick End

### Labeling and Transmission Electron Microscopy

Apoptotic cells were detected by a histochemical staining technique designated terminal deoxyuridine nucleotide end labeling (TUNEL), which permits detection of dead or dying cells (e.g., apoptotic cells) in sectioned tissue, as described in, e.g., Gavrieli et al., J. Cell. Biol. 119:493-501 (1992) or Schulick et al., Proc. Natl. Acad. Sci. USA 95:6983-6988 (1998). The TUNEL method permits in situ labeling of DNA breaks in nucleic, in tissue sections processed through standard histopathological procedures. The method employs terminal deoxynucleotidyl transferase (TdT) to end label DNA fragments within the nucleic of apoptotic cells. TdT specifically binds to the 3′—OH ends of DNA, ensuring a synthesis of a polydeoxynucleotide polymer. After exposure of nuclear DNA on histological sections by proteolytic treatment, TdT is used to incorporate biotinylated deoxyuridine at sites of DNA breaks. The resulting signal is amplified by avidin-peroxidase, enabling conventional histochemical identification by, for example, light microscopy.

With methods according to the present invention, perfusion-fixed vessels embedded in paraffin were cut into sections having a thickness of 5 μm. See, e.g., Schulick et al., Proc. Natl. Acad. Sci. USA 95:6983-6988 (1998). Apoptotic cells were detected by the TUNEL technique, essentially as described in Schulick et al., Proc. Natl. Acad. Sci. USA 95:6983-6988 (1998). Sections of involuting rat mammary gland were stained in parallel with the arterial sections, as positive controls. These sections contained numerous TUNEL-positive, apoptotic cells. Tissue preparation, processing, and transmission electron microscopy (TEM) were performed as reported in Schachtner et al., Circ. Res. 76:701-709 (1995).

### Example 7

### Determination of Vessel Morphometry

Morphometry of vessels, including those vessels used in methods of inducing atherosclerotic lesion development and vessels evaluated for an activity or effect of a test agent on atherosclerotic lesion development, was determined as follows. A computerized morphometric imaging system (Image One, Universal Imaging Corp., West Chester, Pa.) and Movat pentachrome-stained sections from perfusion-fixed arteries were used in histologic analysis of the intimal and medial areas of the vessels and to determine intima area, medial area, and the intimal/medial area ratio of the vessels. The area bounded by the internal and elastic laminae was measured; this is the medial area. The intimal area was calculated by subtraction of the lumen area from the area bounded by the internal elastic lamina. The intimal:medial (I:M) ratio is defined as a ratio of the area of the arterial intima to the area of the arterial media. This ratio functions as a measure of the size of the atherosclerotic lesion. Intimal and medial areas were calculated using data generated by planimetry of the lumenal surface, internal elastic lamina, and external elastic lamina. The percentage of intimal area staining positive for RAM-11 was determined by using the image analysis program to quantitate the area within the intima of RAM-11-stained slides that stained brown with peroxidase reaction product and dividing this area by the total intimal area of the same slides. Results obtained with this technique were highly reproducible. The interobserver correlation (r²) was 0.98. Morphometric results for each artery represented the mean calculated from measurements made on 8 evenly spaced cross sections per vessel, according to the method described in Bolender et al., Am. J. Physiol. 265:L521-L548 (1993). The most proximal and distal of the 10 rings per vessel were excluded from analysis to avoid potential artifacts caused by the vascular clamps and the infusion catheter cannula.

Statistical analyses were performed as follows. Mean intimal/medial area ratios for the various groups of vessels (experimental and control groups) were compared using an unpaired Student's t test. Ranking staining intensities assigned from the immunohistochemical studies were compared by nonparametric analysis. The Mann-Whitney U statistic as in Newman et al., J. Clin. Invest. 96:2955-2965 (1995) was used. See also Mann & Whitney, Ann. Math. Stat. 18:50-60 (1947). Data are expressed herein as median of group. Four sections per vessel were scored by each of the two observers and the median of these eight scores was used to generate a score for the entire vessel. A value of P<0.05 was accepted to denote statistical significance. The SigmaStat Software (Version 2.0, Jandel Scientific, San Rafael, Calif.) was used for the analyses.

### Example 8

### Evaluation of Local Vascular Inflammation and Neointimal

### Formation

Local vascular inflammation and neointimal formation in rabbit vessels (e.g., rabbit carotid arteries) infused with proinflammatory agents (e.g., adenoviral vectors) were identified and evaluated as follows.

The degree of vascular inflammation was determined by evaluating frozen arterial sections immunostained for T cells, VCAM-1, and ICAM-1, as described in detail above. Sections were examined with light microscopy (magnification ×100 and ×400), and the intensity of antibody staining was graded by two independent observers, each blinded to the identity of the sections. Antibody activity on each section was scored with the aid of a semiquantitative scale of 0 to 4 of staining intensity, as described in Newman et al., J. Clin. Invest. 96:2955-2965, 2957 (1995). Specifically, 0=no staining; 1=rare positive cells or staining barely visible at low (×100) magnification power; 2=focal staining or faint diffuse staining clearly visible at low (×100) magnification power; 3=multifocal staining or moderate intensity diffuse staining; 4=intense, diffuse staining.

Neointimal lesions were scored by the same observers using a different semiquantitative scale: 0=no lesion; 1=partial circumference and less than 3 cells thick; 2=partial circumference and greater than 3 cells thick; 3=circumferential lesion. This scoring system was highly reproducible between observers, with over 90% of individual scores given by two observers falling within one point of the scores from the other observer. Four sections per arterial vessel were scored by each of the two observers and the mean of these eight scores was used to generate a score for the entire vessel. The staining intensity and the lesion size scores given by the two observers were highly correlated (r²=0.93 and 0.87 for staining intensity and lesion size, respectively). Vessels harvested at each time point and stained with a particular antibody were scored and ranked in order of intensity of staining. This particular analysis was done at only one time point.

### Example 9

### Neutralizing Anti-Adenoviral Antibody Assay, Complete Blood

### Counts, and Liver Function Tests

Neutralizing serum antibodies to adenovirus type 5 were assayed at 14 days after virus infusion as described in Schulick et at., J. Clin. Invest. 99:209-219 (1997). Complete blood counts and liver function tests [serum alkaline phosphatase, glutamic-oxaloacetic transaminase (SGOT), glutamic-oxalopyruvic transaminase (SGPT) and total bilirubin] were performed by a commercial laboratory (IDEXX, West Sacramento, Calif.).

### Example 10

### Evaluation of Expression of AdFasL and of Cytotoxicity on

### Vascular Cells

Expression of FasL in AdFasL-transduced cells and arteries was evaluated by western blotting (see Kang et al., Nat. Med. 3:738-743 (1997)). Cells and arteries transduced in parallel with AdNull served as controls.

To evaluate FasL-mediated toxicity in vitro, endothelial cells (EC) or SMC were grown to near confluence and infected with AdFasL or AdNull at a concentration of 5×10⁹ particles/ml for 1 hour, and incubated for an additional 12 hours after the addition of either EC or SMC growth media. Mock infections were carried out in parallel by adding an equivalent volume of phosphate-buffered saline (PBS) instead of virus stock. Cells were examined by phase-contrast microscopy for evidence of cytotoxicity and were then harvested and total genomic DNA extracted using the Easy SNAP DNA kit (Invitrogen). Extracted DNA was subjected to electrophoresis on a 1.5% agarose gel, followed by ethidium bromide staining to visualize DNA laddering.

Cell viability and apoptosis were also examined by transducing EC and SMC with AdNull or AdFasL at 1×10⁹ to 9×10¹⁰ particles/ml for 1 hour. Eighteen hours later, the cells were observed by phase contrast microscopy and stained with 5 μM Quinolinium, 4-[(3-methyl-2(3H)-benzoxazolylidene) methyl]-1-[3-(trimethylammonio)propyl]-, diiodide (“YoPro-1”; Molecular Probes, Eugene, Oreg.) for 45 min at 37° C. This dye is taken up only by non-viable cells and stains nuclear DNA. Apoptotic cells were identified by the presence of nuclear condensation and chromatin fragmentation.

### Example 11

### Assay of Mitogenic Activity in Medium Conditioned by Transduced

### Endothelial Cells (EC) or Carotid Arteries

Confluent EC were infected with AdFasL, AdNull, or vehicle alone at a concentration of 5×10⁹ particles/ml for 1 hour. The infection medium was then aspirated and replaced with EC growth medium for an additional 12 hours. The medium was then aspirated, the cells rinsed three times, and the well refilled with M-199 containing 0.4% FCS. Forty-eight hours later, the EC conditioned medium (CM) was collected and stored at −80° C. To generate medium conditioned by rabbit EC transduced in vivo, common carotid arteries were transduced with AdFasL (n=4) or AdNull (n=4) and processed as explants, as described above. As an additional control, two carotid arteries were removed without previous manipulation and placed in explant culture in parallel.

We assayed media conditioned by either cells or arteries for mitogenic activity on rabbit vascular SMC. In this assay, SMC (passage 3-5) were seeded in 96-well plates at a density of 1×10⁴ cells/well, and allowed to attach for 4 hr in M-199 with 10% FCS. The medium was then replaced with M-199 with 0.4% FCS to achieve cellular quiescence. Three days later this medium was aspirated and replaced with either: 1) M-199 with 0.4% FCS (quiescent control); 2) M-199 with 10% FCS (proliferative control); or 3) CM from either cultured EC or ex vivo cultured carotid artery segments. Three days later, cell number was determined in quadruplicate wells using an assay based on the ability of viable cells to convert a tetrazolium salt (MTS) to a colored formazan product (#G5421, Promega). Preliminary control experiments performed with this kit verified a high correlation between the optical density of this converted substrate and the number of SMC in a well, as determined by performing the assay on serially diluted aliquots of SMC (data not shown).

### Example 12

### Statistics

Results are reported as mean±standard error of the mean (SEM) or median and range for data not normally distributed. Normally distributed groups were compared with the unpaired t test. Comparisons between two nonnormally distributed groups were made with the Mann-Whitney rank sum test. Comparisons between multiple groups with normally distributed data were made using one-way ANOVA (i.e., analysis of variants) with controls for multiple pairwise comparisons by the Student-Newman-Keuls method. Results of semiquantitative scoring were compared using the nonparametric Kruskal-Wallis ANOVA with controls for multiple pairwise comparisons by Dunn's method (see Zar, BIOSTATISTICAL ANALYSIS 306-327 (Prentice-Hall, Englewood Cliffs, N.J., 1984). The strength of correlation (r²) between semiquantitative scores of staining intensity given by independent observers was assessed by the Spearman rank order correlation. Differences were considered to be significant if P<0.05.

### DISCUSSION

I. Determining the Effect of Hypercholesterolemia and Adenoviral Vectors on the Development of Carotid Atherosclerosis

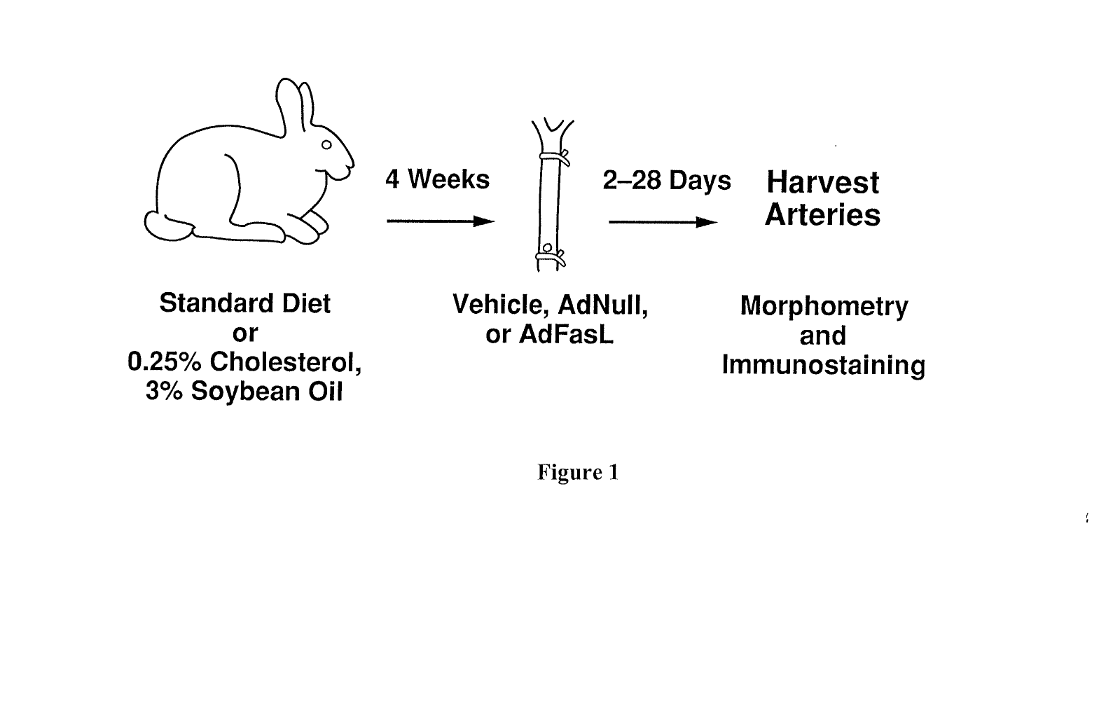

Experiments were performed according to a basic overall design (FIG. 1) in which rabbits were fed either standard or atherogenic diets for four weeks and were then treated with bilateral carotid infusion of either vehicle solution, AdNull, or AdFasL. Arteries were harvested either 14 or 28 days after infusion. The atherogenic diet was administered to induce hypercholesterolemia within a pre-defined range (400-700 mg/dl; see above discussion). Notably, rabbits fed this (or other atherogenic) diet(s) for four weeks did not develop carotid artery intimal lesions (n=4; data not shown). Thus, our experiments investigate lesions that form after the vectors (or vehicle solution) have been infused.

In the first series of experiments, vehicle solution was compared to AdNull in the presence and absence of hypercholesterolemia. In the second series of experiments, AdNull was compared to AdFasL in the presence of hypercholesterolemia. In the first series of experiments, plasma cholesterol in the vehicle infusion+atherogenic diet group was 598±63 mg/dl at the time of carotid infusion and 454±41 at the 4 week artery harvest. The corresponding plasma cholesterol levels in the AdNull+atherogenic diet group were 561±46 and 582±155 mg/dl (P>0.5 versus the vehicle group at both time points). AdFasL-infused rabbits fed the atherogenic diet had corresponding cholesterol levels of 552±172 and 952±125 mg/dl (P=0.78 and 0.1 for AdFasL versus AdNull at the two time points). Rabbits fed the normal diet had cholesterol levels of 27±2.5 mg/dl.

Infusion of AdNull or vehicle in the presence or absence of hypercholesterolemia produced four strikingly different arterial morphologies at 4 weeks after infusion. First, arteries infused with vehicle in the absence of hypercholesterolemia appeared essentially normal (FIGS. 2A and 2E), establishing that vehicle infusion alone did not cause intimal lesion formation. Second, arteries infused with vehicle in the presence of hypercholesterolemia contained foamy intimal lesions (FIG. 2B) composed of macrophages (identified with RAM-11: FIG. 2F) and SMC (identified with HHF-35; data not shown). These lesions were found focally along the length of the artery and were non-circumferential within individual sections. Vessels in this group harvested at 14 days after vehicle infusion contained macrophages adherent to and below intact endothelium and a smaller, SMC-containing intima (data not shown). Third, arteries infused with AdNull in the absence of hypercholesterolemia had focal, eccentric, non-foamy lesions that were highly cellular but did not contain macrophages (FIGS. 2C and 2G). Fourth, arteries infused with AdNull in the presence of hypercholesterolemia contained larger lesions that were rich in foam cells and macrophages (FIGS. 2D and 2H). The luminal endothelium was intact in all arteries in all four groups. The intimal histology in the four groups was sufficiently distinct and non-overlapping that a blinded observer placed 100% of the experimental arteries in the correct treatment group simply by examining several histologic sections from each artery.

Quantitative morphometric analyses performed on sections from arteries harvested 4 weeks after infusion of vehicle or virus supported the impressions derived from the histologic sections. Arteries from normocholesterolemic rabbits infused with vehicle (n=7) had an I/M ratio of 0.07±0.01 (FIG. 3A). Because the intima in these arteries was composed almost exclusively of a single layer of endothelium, this figure (0.07±0.01) approximates the I/M ratio for a normal, nondiseased rabbit carotid artery, determined by our technique of planimetry. The I/M ratio for arteries from hypercholesterolemic rabbits infused with vehicle (n=6) was 0.08±0.01 (P>0.5 versus vehicle solution, normal, standard diet). Thus, despite the presence of small intimal lesions in arteries from the hypercholesterolemic rabbits, and their absence in arteries from normocholesterolemic rabbits, the I/M ratios of the two groups were nearly identical. The failure of planimetry to separate these two groups is likely due to the insensitivity of this technique to detect an increase in I/M ratio caused by lesions that are relatively small and eccentric. However, these two groups were easily differentiated based both on histologic findings (see above) and by use of a semiquantitative method for scoring lesion size (see below). The I/M ratio of arteries from normocholesterolemic rabbits infused with AdNull was 0.15±0.02 (n=8; P<0.05 versus vehicle solution only). Arteries exposed to both hypercholesterolemia and AdNull infusion had a mean I/M ratio of 0.28±0.02 (n=7; P<0.05 versus both AdNull alone and vehicle solution alone). Thus, the morphometric data confirmed that hypercholesterolemia and adenovirus make distinct, additive contributions to lesion formation.

To quantitate the contribution of macrophage infiltration to intimal lesion formation in the four experimental groups, we measured the percentage of intimal area staining positively for the macrophage marker RAM-11 (FIG. 3B). Macrophages were present only in arteries from hypercholesterolemic rabbits and represented a significantly larger percentage of lesion area in AdNull-infused than in vehicle-infused arteries (29±5 versus 2.4±1.5%; P<0.05). Thus, adenovirus accelerates both the rate of lesion growth and the rate of macrophage accumulation.

We hypothesized that adenovirus might promote lesion progression by enhancing vascular cell activation and arterial wall inflammation. Nevertheless, it was possible that in a setting conducive to cholesterol-induced vascular activation (Li et al., Arterioscler. Thromb. 13:197-204 (1993), infusion of adenovirus would make no additional contribution to arterial wall inflammation. To address this issue, we harvested arteries at 14 days after vehicle or AdNull infusion, a time at which preliminary data showed that vascular inflammation was well developed and lesions had begun to form (data not shown). Significant T cell infiltrates and expression of VCAM-1 and ICAM-1 were found in all arteries infused with AdNull, both in the presence and absence of hypercholesterolemia (data not shown). The positively stained areas occupied a smaller percentage of the lesions in arteries from hypercholesterolemic rabbits (data not shown), most likely because lesions from hypercholesterolemic rabbits were larger and contained substantial numbers of macrophages (which did not stain with the antibodies to CD5, VCAM-1, and ICAM-1; data not shown). In contrast to the adenovirus-infused arteries, vehicle-infused arteries contained essentially no T cells and no VCAM-1 expression (see FIGS. 4A-4D and data not shown). ICAM-1 expression, however, was detected even in the vehicle-infused arteries, although at a lower level than in the AdNull-infused arteries. Semi-quantitative analysis of staining intensity (FIGS. 4A-4C) revealed that T cells were present and both VCAM-1 and ICAM-1 were expressed at similar levels in the AdNull-transduced arteries both in the presence and absence of hypercholesterolemia.

We also evaluated lesion size in immunostained sections from the 14 day arteries. Because these arteries were not perfusion fixed and because their lesions were relatively small, lesion size was evaluated with a semiquantitative scale (see Methods). Consistent with our other data (FIGS. 2A-2H and FIGS. 3A and 3B), arteries from normolipidemic rabbits that were infused with vehicle did not have intimal lesions (FIG. 4D). In contrast, vehicle-infused arteries from hypercholesterolemic rabbits demonstrated significant lesion formation (P<0.05 versus vehicle solution, no cholesterol). AdNull-infused arteries from both hypercholesterolemic and normolipidemic rabbits had even larger intimal lesions (P<0.001 for both groups versus vehicle solution, no cholesterol).

In summary, surgical manipulation and vehicle infusion into carotid arteries of hypercholesterolemic rabbits caused early intimal atherosclerotic lesions that—at 4 weeks after infusion—were composed largely of macrophages and SMC. Infusion of AdNull instead of vehicle caused T cell infiltrates and vascular cell activation and increased both the macrophage and SMC components of the intimal lesions. The intimal lesions developed below morphologically intact endothelium. Thus, the pathology that resulted from adenovirus infusion in the arteries of hypercholesterolemic rabbits mimicked the early development of human atherosclerosis (Ross, Nature 362:801-809 (1993); Stary et al., Circulation 89:2462-2478 (1994)) and did so far better than does hypercholesterolemia alone.

II. Murine FasL Causes Apoptosis of Rabbit Vascular SMC in Vitro and Can Be Expressed in Rabbit Endothelium in Vivo

Rabbit vascular SMC were exposed to either AdNull or AdFasL at a concentration of 5×10⁹ particles/ml. Cells exposed to AdNull remained intact and adherent (FIG. 9A) and could not be distinguished from mock-transduced cells exposed in parallel to vehicle solution only (not shown). In contrast, most of the cells exposed to AdFasL appeared pyknotic (FIG. 9B). Agarose electrophoresis of DNA harvested from these cells revealed laddering characteristic of apoptosis only in the AdFasL-transduced cells (FIG. 9C). Thus, murine FasL triggered apoptosis in rabbit vascular cells. Next, we tested whether AdFasL directed expression of FasL protein in vivo. Western blot analysis detected a protein of approximately 40 kiloDalton (kDa) in extracts of AdFasL but not AdNull-transduced arteries (n=2 per group; data not shown).

III. Effects of AdFasL on Humoral Immune Response, T Cell Infiltration, Vascular Activation, Lesion Size, and Macrophage Content

We used the basic experimental design (FIG. 1) to investigate the role of endothelial FasL expression in atherosclerotic lesion development. Arteries in hypercholesterolemic rabbits were transduced with either AdNull or AdFasL and harvested from 2 days to 4 weeks later. Because systemic delivery of AdFasL can cause lethal hepatic apoptosis (Muruve et al., Hum. Gene Ther. 8:955-963 (1997)), complete blood counts and liver function tests were performed at 7, 14, and 28 days after vector infusion. These tests revealed no difference between the AdNull and AdFasL groups and no evidence of systemic toxicity (data not shown).

Expression of FasL had pleiotropic effects on the immune and inflammatory responses to adenovirus infusion. AdNull and AdFasL produced similar humoral immune responses to adenoviral proteins: rabbits infused with either vector all developed high serum titers of anti-adenoviral antibodies (≧1:512), with no significant difference between the two groups. As expected, AdNull-transduced arteries harvested at 14 days (n=6) showed robust infiltration of T cells and expression of ICAM-1 and VCAM-1 (FIGS. 10A-10C). In contrast, 14 day FasL-transduced arteries (n=5) had significantly fewer T cells in the intima or media and essentially no expression of VCAM-1 (FIGS. 10D and 10E; FIGS. 11A and 11B; P<0.05 for CD5 and VCAM-1 scores). ICAM-1 expression was detectable in FasL-transduced arteries at a level similar to that seen in arteries transduced in parallel with AdNull (FIG. 10C, FIG. 10F, and 11C). Despite the paucity of T cells and the marked decrease in vascular inflammation, intimal lesions were present in all AdFasL-transduced arteries (FIGS. 10D-10F). Indeed vascular lesions were larger in the AdFasL arteries (FIG. 11D; P<0.05).

Planimetry of cross sections of arteries perfusion fixed in situ and harvested at 7, 14, and 28 days confirmed that FasL gene transfer accelerated neointimal formation (FIG. 5). At 7 days, the I/M ratio of FasL-transduced arteries was 3-fold greater than the AdNull-transduced arteries. This difference persisted at 14 days arteries, but was no longer present at 28 days.

To investigate the cellular mechanisms by which FasL accelerated lesion formation, we stained the lesions with cell type-specific antibodies and with a histochemical stain for neutrophils. The increase in lesion size at 7 and 14 days was not due primarily to accelerated macrophage accumulation because the percentage of RAM-11-stained area was the same in arteries transduced with either AdFasL or AdNull (FIG. 6 and other data not shown). We considered that AdFasL might stimulate neointimal growth as a consequence of EC apoptosis. However, staining for the endothelial marker CD-31, as well as TUNEL staining performed on arteries transduced with either AdFasL or AdNull and harvested 2 or 7 days later, revealed nearly intact luminal endothelial monolayers (95-100% intact endothelium in all arteries from both groups) with no significant apoptosis (FIGS. 7A and 7B and data not shown). We also postulated that vascular injury potentially resulting from FasL-induced apoptosis of medial SMC might stimulate neointimal formation. However, all 7 and 14 day AdFasL and AdNull arteries had an intact SMC-containing media with no significant TUNEL positivity (FIGS. 7A-7B, FIGS. 8A-8B and data not shown). Finally, we considered whether the neointima in AdFasL-transduced arteries might consist largely of neutrophils, as neutrophilic infiltrates are associated with ectopic FasL expression (Kang et al., Nat. Med. 3:738-743 (1997); Allison et al., Proc. Natl. Acad. Sci. USA 94:3943-3947 (1997)). However, the neointimas did not contain neutrophils, based on evaluation with both H & E and Leder stains (not shown).

IV. Nature of Intimal Lesions Induced by FasL

We considered that the majority of these intimal cells might be smooth muscle actin-negative vascular SMC (i.e., the synthetic, proliferative phenotype (Schwartz et al., Circ. Res. 77:445-465 (1995); Ross, Annu. Rev. Physiol. 57:791-804 (1995). Examination of ultrathin sections by TEM supported this hypothesis, because the majority of intimal cells had features suggestive of the synthetic SMC phenotype: abundant rough endoplasmic reticulum and surrounding collagen fibrils (FIGS. 8A-8B).

To investigate the contribution of enhanced proliferation to the accumulation of intimal SMC, we stained sections from 2 and 7 day arteries with an antibody to PCNA. Intimal lesions at 2 days were rare and small in all arteries, with only occasional cells staining positively for PCNA (not shown). Intimal cells in both the 7 day AdNull and AdFasL-transduced arteries showed PCNA expression (data not shown). However, the percentage of intimal cells expressing PCNA was significantly higher for AdFasL-transduced arteries (37±7.5% versus 15±6.3%; P<0.05). Medial SMC in these arteries demonstrated only rare PCNA staining (data not shown).

Taken together, these morphological and histochemical analyses show that FasL expression by arterial endothelium did not cause cell death or apoptosis of EC or SMC in vivo. Neither did FasL expression act in a pro-inflammatory manner: T cell infiltrates and VCAM-1 expression were decreased in FasL-transduced arteries, macrophage abundance was unaffected, and neutrophils were absent. Rather, FasL expression increased the rate of development of intimal lesions at least in part by promoting intimal SMC proliferation. Notably, at the time points examined, the mitogenic effect of FasL expression was restricted to intimal cells (data not shown).

V. In Vitro Investigations of Mechanism of Vascular Effects of AdFasL

We sought to establish a cell culture model to investigate the mechanisms through which endothelial FasL expression produces its in vivo effects (i.e., no apoptosis of EC or SMC with proliferation of intimal SMC). To begin these investigations, we transduced cultured rabbit SMC and EC with AdFasL at concentrations ranging from 1×10⁹ to 9×10¹⁰ particles/ml. Examination of SMC by phase contrast microscopy 18 hrs later revealed dose-related toxicity with gradually increasing amounts of detachment and pyknosis (FIG. 12A). To determine whether these SMC were apoptotic, we stained them with “Yo-Pro-1”, a DNA-binding dye to which viable cells are impermeable. The dye was absorbed by most of the floating cells as well as some of the attached cells. In almost all cases the pattern of fluorescent dye accumulation was characteristic of apoptosis (fragmented, marginated chromatin; FIG. 12B). In contrast, EC exposed to AdFasL at 1×10⁹ to 3×10¹⁰ particles/ml remained in intact monolayers (FIG. 12C). Even when rare endothelial cells (EC) did take up the dye, they showed only diffuse nuclear fluorescence, suggestive of a nonapoptotic death (FIG. 12D). Thus, cultured rabbit EC were resistant to FasL-mediated apoptosis and are an appropriate model with which to investigate the resistance of rabbit arterial endothelium in vivo to FasL-mediated apoptosis. (FIGS. 7A-7B).

The in vivo mitogenic effect on intimal SMC of endothelial FasL expression, however, is not accurately modeled by the above in vitro experiment in which SMC are reliably killed by exposure to FasL. We hypothesized that FasL-transduced EC, while resistant to FasL-mediated apoptosis, might secrete a mitogen to which intimal SMC are receptive. To test this hypothesis, we transduced EC both in vitro and in vivo with AdFasL and tested EC-conditioned media for mitogenic activity on cultured rabbit SMC. Media conditioned by mock-transduced cultured EC had a low level of mitogenic activity: SMC treated with this CM were 1.8-fold more numerous than control SMC maintained in 0.4% FCS (n=12 samples in 2 independent experiments; range 1.7-2.1). Mitogenic activity in CM from EC transduced in parallel with AdNull was essentially the same (1.9-fold increase in SMC number above controls; range 1.6-2.1). Mitogenic activity in CM from EC transduced with AdFasL was not increased above this level and was toxic to SMC in one of the two independent experiments (mean 1.4-fold increase in SMC number above controls; range 0.22-2.2). We extended these studies by transducing arteries in vivo with AdNull (n=4) and AdFasL (n=4) and explanting the arteries after 24 hours. Medium conditioned by these arteries for 48 hrs ex vivo was tested for mitogenic activity on SMC. Control CM from two unoperated arteries had no detectable mitogenic activity (mean 1.1-fold increase in SMC number; essentially equivalent to 0.4% FCS). CM from AdNull and AdFasL-transduced arteries had equivalent mitogenic activity (both produced mean 1.4-fold increases in SMC number, ranges 1.2-1.4 and 1.1-1.5-fold, respectively). Within the sensitivity of our assays, we were unable to detect a FasL-mediated increase in EC-derived SMC mitogens.

In summary, the present invention provides methods for screening test agents for an activity on atherosclerotic lesion development in a non-human mammal. In particular, the present invention provides methods of screening genetic agents, such as genes, for a therapeutic or prophylactic activity on atherosclerotic lesion development in a non-human mammal. The present invention also provides non-human mammalian models which are representative of human atherosclerosis and its conditions. In addition, the present invention provides methods of producing such models of atherosclerosis. With the inventions presented herein, fundamental questions about the pathogenesis of atherosclerosis and related vascular diseases can be addressed. Effective strategies for the prevention and treatment of such diseases in humans can be developed by using the inventions disclosed herein.

As will be apparent to those of ordinary skill in the art based on the detailed disclosure provided herein, the methods of the present invention are broadly applicable to other aspects of animal models of atherosclerosis and pathogenesis of atherosclerosis and to other aspects of in vivo and ex vivo screening methods and prophylactic and therapeutic gene therapy methods. The animal models of the present invention can be used in other methods for screening agents for effects on vascular diseases. Such other animal models, screening methods and gene therapy methods can be used in a variety of applications, including those discussed above.

While the foregoing invention has been described in some detail for purposes of clarity and understanding, it will be clear to one skilled in the art from a reading of this disclosure that various changes in form and detail can be made without departing from the true scope of the invention. The above examples are provided to illustrate the invention, but not to limit its scope; other variants of the invention will be readily apparent to those of ordinary skill in the art and are encompassed by the claims of the invention. The scope of the invention should, therefore, be determined not with reference to the above description, but instead should be determined with reference to the appended claims along with their full scope of equivalents. All publications, references, and patent documents cited in this application are incorporated by reference in their entirety for all purposes to the same extent as if each individual publication or patent document were so individually denoted.

What is claimed is:

1. A method of screening a test agent for an activity on atherosclerotic lesion development in a non-human mammal, the method comprising:
(a) administering to the non-human mammal an atherogenic diet;
(b) isolating a blood vessel of the non-human mammal;
(c) delivering a proinflammatory agent to the blood vessel of the non-human mammal;
(d) delivering the test agent to the non-human mammal; and
(e) monitoring a property of the blood vessel to indicate an activity on atherosclerotic lesion development in the blood vessel.

2. The method of claim 1, wherein the blood vessel is an artery.

3. The method of claim 1, wherein the blood vessel is a vein.

4. The method of claim 2, wherein the activity is an increase in atherosclerotic lesion development.

5. The method of claim 2, wherein the activity is a decrease in atherosclerotic lesion development.

6. The method of claim 5, wherein the proinflammatory agent comprises a vector, cytokine, or chemokine.

7. The method of claim 6, wherein the proinflammatory agent is a vector.

8. The method of claim 7, wherein the vector is a viral vector.

9. The method of claim 8, wherein the viral vector comprises an adenoviral vector, retroviral vector, adenoassociated viral vector, alphaviral vector, or herpes simplex viral vector.

10. The method of claim 9, wherein the viral vector is an adenoviral vector.

11. The method of claim 7, wherein the vector comprises the test agent.

12. The method of claim 11, wherein the test agent comprises a gene which is expressed in the artery upon delivery of the vector to the artery.

13. The method of claim 12, wherein the gene encodes a therapeutically or prophylactically useful protein or peptide for treating atherosclerosis or atherosclerotic lesion development.

14. The method of claim 7, wherein the vector comprises a gene which encodes the test agent, wherein said gene expresses the test agent in the artery upon delivery of the vector to the artery.

15. The method of claim 14, wherein the test agent encoded by the gene comprises a therapeutically or prophylactically useful protein or peptide for treating atherosclerosis or atherosclerotic lesion development.

16. The method of claim 1, wherein the test agent comprises a nucleic acid segment, gene, pharmaceutical compound, toxin, natural product, or chemical compound being screened for an activity on atherosclerotic lesion development.

17. The method of claim 16, wherein the test agent is a gene being screened for an activity on atherosclerotic lesion development.

18. The method of claim 2, wherein the blood vessel comprises a carotid artery, femoral artery, brachial artery, radial artery, gastroepiploic artery, iliac artery, innominate artery, aorta, coronary artery, or vertebral artery.

19. The method of claim 5, wherein the property of the artery being monitored is an arterial property, said arterial property comprising an increase in inflammation in the blood vessel.

20. The method of claim 5, wherein the property of the artery being monitored is an arterial property, said arterial property comprising an increase in smooth muscle cells in an intima of the artery.

21. The method of claim 5, wherein the property of the artery being monitored is an arterial property, said arterial property comprising an increase in T cells or macrophages in an intima of the artery.

22. The method of claim 5 wherein the property of the artery being monitored is an arterial property, said arterial property comprising an increase in the volume of an intima of the artery, a narrowing of a lumen of the artery, a decrease in blood flow through the artery, an increase in lipid in a wall of the artery, an accumulation of extracellular matrix in a wall of the artery, or a rupture of a plaque in the artery.

23. The method of claim 1, wherein the proinflammatory agent and test agent are delivered to the non-human mammal simultaneously.

24. The method of claim 1, wherein the test agent is delivered to the non-human mammal before the proinflammatory agent is delivered to the non-human mammal.

25. The method of claim 1, wherein the non-human mammal comprises a rabbit, mouse, pig, or non-human primate.

26. The method of claim 6, wherein the non-human mammal is a rabbit and the artery is a carotid artery.

27. A method of screening a gene for a therapeutic or prophylactic activity on atherosclerotic lesion development in a non-human mammal, the method comprising:
(a) administering an atherogenic diet to the non-human mammal;
(b) isolating a blood vessel of the non-human mammal;
(c) delivering a vector to the blood vessel, wherein the vector comprises a gene and expresses the gene in the blood vessel; and
(d) monitoring a property of the blood vessel which indicates a therapeutic or prophylactic activity of the gene on atherosclerotic lesion development in the blood vessel.

28. The method of claim 27, wherein the therapeutic or prophylactic activity is anti-atherosclerotic activity.

29. The method of claim 28, wherein the anti-atherosclerotic activity is a decrease in atherosclerotic lesion development.

30. A method of producing a non-human mammalian model of atherosclerosis, the method comprising:
(a) administering an atherogenic diet to the non-human mammal;
(b) isolating a blood vessel of the non-human mammal;
(c) delivering a proinflammatory agent to the blood vessel of the non-human mammal; and
(d) maintaining the non-human mammal for a time sufficient for an atherosclerotic lesion to develop in the blood vessel, thereby producing a model of atherosclerosis.

31. The method of claim 30, wherein the proinflammatory agent comprises a vector, cytokine, or chemokine.

32. The method of claim 31, wherein the proinflammatory agent is a vector.

33. The method of claim 32, wherein the vector is a viral vector.

34. The method of claim 33, wherein the viral vector comprises an adenoviral vector, retroviral vector, adenoassociated viral vector, alphaviral vector, or herpes simplex viral vector.

35. The method of claim 34, wherein the vector is an adenoviral vector.

36. The method of claim 35, wherein the non-human mammal comprises a rabbit, mouse, pig, or non-human primate.

37. The method of claim 36, wherein the non-human mammal is a rabbit.

38. The method of claim 37, wherein the blood vessel comprises an artery, wherein said artery comprises a carotid artery, femoral artery, brachial artery, radial artery, gastroepiploic artery, iliac artery, innominate artery, aorta, coronary artery, or vertebral artery.

39. A non-human mammalian model for atherosclerotic disease, wherein the model comprises a non-human mammal having a blood vessel, said blood vessel characterized by having an atherosclerotic lesion, wherein the blood vessel has an intact endothelium and an intima comprising smooth muscle cells, and the atherosclerotic lesion includes a proinflammatory agent.

40. The non-human mammalian model of claim 39, wherein the intima further comprises T cells or macrophages.

41. The non-human mammalian model of claim 39, wherein the proinflammatory agent is a viral vector.

42. A method of inducing development of an atherosclerotic lesion in a non-human mammal, the method comprising:
(a) administering to the non-human mammal an atherogenic diet;
(b) isolating a blood vessel of the non-human mammal; and
(c) introducing a proinflammatory agent into the blood vessel, wherein said administering to the non-human mammal of the atherogenic diet and said introducing of the proinflammatory agent into the blood vessel of the non-human mammal induces or causes development of an atherosclerotic lesion in the blood vessel.

43. A method of gene therapy for prophylactic or therapeutic treatment of atherosclerosis in a subject in need of such treatment, which comprises delivering a vector to a blood vessel of the subject, wherein said vector comprises a gene which encodes a therapeutically or prophylactically useful peptide or protein for atherosclerosis, said gene being expressed in the subject, thereby promoting prophylactic or therapeutic treatment of atherosclerosis in the subject.
